(19) United States
(12) Patent Application Publication
(10) Pub. No.: US 2026/0115325 A1
(43) Pub. Date: Apr. 30, 2026

(54) METHODS AND COMPOSITIONS FOR TREATING CORNEAL WOUNDS

(71) Applicant: Northwestern University, Evanston, IL (US)

(72) Inventors: Han Peng, Evanston, IL (US); Robert M. Lavker, Evanston, IL (US)

(21) Appl. No.: 19/367,578

(22) Filed: Oct. 23, 2025

Related U.S. Application Data
(60) Provisional application No. 63/711,554, filed on Oct. 24, 2024.

(51) Int. Cl. A61K 48/00; A61K 31/7105; A61K 38/17; A61P 27/02; C12N 15/113; C12N 15/86

(52) U.S. Cl. CPC A61K 48/0058; A61K 31/7105; A61K 38/177; A61P 27/02; C12N 15/113; C12N 15/86; C12N 2310/14; C12N 2310/531; C12N 2750/14143

(57) ABSTRACT

Methods and compositions for treating a corneal wound and/or increasing a population of limbal epithelial stem cells (LESCs) and/or early transit amplifying cells (eTAs) in the limbal epithelium of a subject in need thereof. The methods can include administering a therapeutic agent to the subject that: i) results in an increase in concentration of IFITM1 (Interferon Induced transmembrane Protein 1) in the limbal epithelium of the subject; and/or ii) results in a decrease in concentration of OVOL1 (Ovo Like Zinc Finger 1) in the limbal epithelium of the subject. Also provided are adeno-associated viruses (AAVs). The AAVs comprise a heterologous nucleic acid sequence encoding IFITM1 or a variant thereof, operably linked to a regulatory sequence.

### CROSS-REFERENCE TO RELATED PATENT APPLICATIONS

This application claims priority to U.S. Provisional Application No. 63/711,554, filed on Oct. 24, 2024. The content of which is herein incorporated by reference in its entirety.

### STATEMENT REGARDING FEDERALLY SPONSORED RESEARCH OR DEVELOPMENT

This invention was made with government support under grant numbers EY028560 and EY032922 awarded by the National Institutes of Health. The government has certain rights in the invention.

### REFERENCE TO AN ELECTRONIC SEQUENCE LISTING

A Sequence Listing accompanies this application and is submitted as an XML file of the sequence listing named “10_22_25_702581.02724_SequenceList.xml” which is 149,659 bytes in size and was created on Oct. 22, 2025. The sequence listing is electronically submitted via Patent Center with the application and is incorporated herein by reference in its entirety.

### FIELD

The field of the invention relates to methods and compositions for treating corneal wounds.

### BACKGROUND

The corneal epithelium is a self-renewing stratified squamous epithelium. A unique feature of the corneal epithelium is that the stem cells, which govern self-renewing tissues, are not located in the corneal epithelium; rather they are situated in the basal layer of the limbal epithelium (called limbal epithelial stem cells (LESCs)). LESCs possess stem cell properties (“stemness”) such as: quiescence, extensive proliferative capacity, expression of putative stem cell markers (e.g., Gpha2), and can generate committed progeny: transit amplifying (TA) cells. Early TA cells (eTA), which represent immediate stem cell progeny, also reside in the limbal epithelial basal layer and have high proliferative capacity.

### SUMMARY

In one aspect, a method for treating a corneal wound in a subject in need thereof is provided. The method can include administering a therapeutic agent to the subject that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

In another aspect, a method for increasing a population of limbal epithelial stem cells (LESCs) and/or early transit amplifying cells (eTAs) in the limbal epithelium of a subject in need thereof is provided. The method can include administering a therapeutic agent to the subject that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

In another aspect, the disclosure provides an adeno-associated virus (AAV). The AAV comprises a heterologous nucleic acid sequence encoding IFITM1 or a variant thereof, operably linked to a regulatory sequence(s) which directs expression of the IFITM1 or the variant thereof. In some embodiments, the IFITM1 or variant thereof has an amino acid sequence that having at least 85%, 90%, 95%, 96%, 97%, 98%, 99%, or 100% identity to SEQ ID NO: 1, and/or the AAV comprises a heterologous nucleic acid sequence having at least 85%, 90%, 95%, 96%, 97%, 98%, 99%, or 100% identity to SEQ ID NO: 4.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B. scRNA-seq demonstrated 15 clusters in cells isolated from limbal/corneal tissues of uninjured and NaOH injured mice. (1A) UMAP visualized 15 differential clusters of cells. (1B) Dot plot was used to visualize conserved cell type markers and show both the expression level and the percentage of cells in a cluster expressing those marker genes. For example, Glycoprotein Hormone Subunit Alpha 2(Gpha2) is a limbal epithelial stem/eTA cell marker and cluster 0 which highly expressed Gpha2 was identified as stem/early TA cells.

FIGS. 2A-2D. IFITM1 is predominately expressed in stem/early TA cells, and its expression is positively associated with expansion of stem/early TA cell population after injury. (2A) UMAP visualized 15 differential clusters of cells in each sample. Blue circle highlighted stem/early TA cell population and visualized the cell number of stem/eTA cell population was increased in NaOH injured WT cornea compared to in uninjured WT cornea. (2B) Violin plots show the expression of the gene Ifitm1 among epithelial clusters. Among epithelial cell clusters, Ifitm1 was highly expressed in the limbal stem/eTA cell cluster. (2C) Violin plots show that the expression of Ifitm1 was markedly increased in limbal stem/eTA cells by NaOH injury compared to the uninjured controls. (2D) Immunofluorescent images visualized the expression of Ifitm1 in limbal epithelium (Krt15+ cells) and showed that Ifitm1 proteins were detected in cell nuclei and the relative fluorescent levels for Ifitm1 were markedly increased in NaOH injured mouse limbal epithelium (N=13) compared to uninjured control (N=11). *: p<0.05.

FIGS. 3A-3D. Knockdown of Ifitm1 reduces the cell number of stem/early TA cell population after NaOH injury. Wild type mouse eyes were topically treated with AAV-shRNA-Ifitm1 (SEQ ID NO: 6) or AAV-empty vector (control) and subsequently were subjected to a corneal NaOH burn. (3A) Immunofluorescent images visualized the expression of Ifitm1 in limbal epithelium (Krt15+ cells) and showed that Ifitm1 proteins were markedly decreased in limbal epithelium of mice topically applied with AAV-shRNA-Ifitm1 (N=3) compared to AAV empty vector (control; N=3). (3B) 3 days post injury, single cell RNA sequencing was conducted and UMAP visualized differential clusters of cells from mice with 3 different treatments: uninjured, NaOH injury+AAV-empty vector, and NaOH injury+AAV-shRNA-Ifitm1. Red dots highlights the stem/eTA cell population. (3C) Counting the cell numbers of stem/eTA cell population showed that NaOH injury increased the cell numbers of stem/eTA cells while such increase was reversed by topical treatment of AAV-shRNA-Ifitm1. N=2. *: p<0.05. (3D) RT-qPCR revealed a significant increase in IFITM1 expression in mouse corneas after NaOH exposure compared with uninjured corneas. *p<0.05.

FIGS. 4A-4B. Knockdown of IFITM1 reduces limbal epithelial cell proliferation. HLECs (4A, N=6) and a limbal epithelial cell line hTCEpi cells (4B, N=3) were transfected with siControl or siIFITM1 for 72 hours. Immunofluorescent images visualized the BrdU+ cells at 72 h after transfection. The percentage of BrdU+ cells were quantified by Image J and the results showed a decreased percentage of BrdU+ cells after siIFITM1 transfection. *p<0.05.

FIGS. 5A-5H. Knockdown of Ifitm1 in vivo reduces limbal epithelial cell proliferation. Mouse ocular surfaces were topically treated once with AAV-shRNA-Ifitm1 or AAV empty vector (control). Fourteen days after AAV application, corneas were subjected to NaOH injury (5A, 5C, 5E, 5G) or debridement wounding (5B, 5D, 5F, 5H). (5A, 5B) 24 hours after injury, immunofluorescent images showed that BrdU+ cells in the limbal epithelium of mice with AAV-shRNA-Ifitm1 (N=13) were decreased compared to control (N=10). (5E, 5F) The BrdU+ cells were counted using ImageJ. *P<0.05. (5C, 5D) 24 hours after injury, immunofluorescent images showed that Ki67+ cells in the limbal epithelium of eyes with AAV-shRNA-Ifitm1 (N=3) were decreased compared to control (N=3). (5G, 5H) The Ki67+ cells were counted using ImageJ. *P<0.05.

FIGS. 6A-6E. IFITM1 positively regulates limbal epithelial proliferation via OVOL1. (6A) Immunofluorescent staining showed NaOH burn in mouse corneas reduced Ovol1 in limbal epithelium compared to uninjured mice. Relative fluorescence intensity was analyzed using Image J. *p<0.05. (6B and 6C) Cells were transfected with siControl, siIFIMT1, siOVOL1 or siIFIMT1+siOVOL1 for 72 hours. The percentage of BrdU+ cells were quantified by Image J. *p<0.05. (6D) Immunofluorescent staining showed an increase in Ovol1 expression in the limbal epithelium of mice treated with AAV-shRNA-Ifitm1 compared with AAV-EV control mice. (6E) Relative fluorescence intensity was analyzed using Image J. *p<0.05. N=6. pc: peripheral cornea.

FIGS. 7A-7F. Bulk RNA sequencing identifies DEGs in HLECs transfected with siIFITM1. (7A) HLECs were transfected with siIFITM1 or sicontrol. (7B) PCA plot of bulk RNA-seq from HLECs transfected with siIFITM1 (blue; n=3; from left to right: S44069siIFITM1_S2, S20230202siIFTM1_S7, S43990siIFTM1_S4) and sicontrol (red; n=3; from left to right: S44069sicontrol_S1, S20230202sicontrol_B_S6, S43990sicontrol_S3). *p<0.05. (7C) Heatmap of top 50 differentially regulated genes. Listed from top to bottom, the Groups are: CYP4F22, PRR9, LCE2C, HCN2, PALM, TNNI2, HS6ST3, REEP1, MIR6510, MYPN, CD180, CLDN6, PTPN5, PLIN5, TEK, GABRR2, IGLF1, LINC0248, TUBB2B, RAB39B, HES7, PSTPIP1, SLC45A1, CTNND2, LINC0191, KCNT2, IL21R, DNAJC12, CXCL9, CCDC184, CCDC136, TCEAL7, SEMA3D, IFITM1, DCHS2, CPT1C, KIF5C, PLA2G3, SYT11, GNAZ, SLC8A1, ODAD2, APOL4, FSD1, FABP4, GBX2, NEFL, CA10, NLGN4X, ADGRL3. (7D) Network analysis of scRNA-seq data predicts the connection between Ifitm1 and Ovol1 using Genemania. This suggests that IFITM1 may connect with Ovol1 through Ifitm2/Prlr/Limk2 and/or Spag7/Dctn6. Yellow circles: DEGs found in our bulk RNA seq for siIFITM1 vs siControl. (7E) Among 3 DEGs (LIMK2, IFITM2, and DCTN6), RT-qPCR revealed a significant reduction of DCTN6 expression in HLECs cells transfected with siIFITM1 compared with siControl. N=3 independent experiments. *p<0.05. (7F) RT-qPCR revealed that transfection of siDCTN6 significantly reduced DCTN6 expression while increased OVOL1 in HLECs cells compared with siControl. N=3 independent experiments. *p<0.05.

FIGS. 8A-8D. Bulk RNA sequencing shows that OVOL1 is upregulated in HLECs transfected with siIFITM1. (8A) Volcano plot showed DEGs with more than 2 fold changes between siIFITM1 and siControl treated HLECs. Blue arrow pointed out OVOL1 was upregulated in HLECs transfected with siIFITM1. (8B) Gene ontology (GO) analysis of DEGs was conducted between siIFITM1 and siControl treated HLECs. GO analysis showed that IFITM1 played a role in cell proliferation. (8C) RT-qPCR revealed a significant reduction of IFITM1 gene expression and increase of OVOL1 gene expression in HLECs cells transfected with siIFITM1 compared with siControl. N=3 independent experiments. *p<0.05. (8D) RT-qPCR results revealed a significant reduction of OVOL1 gene expression in HLECs cells after siOVOL1 transfection compared with siControl transfection. N=3 of independent experiments. *p<0.05.

FIGS. 9A-9B. Depletion of Ifitm1 does not result in alteration of cell viability. Mouse ocular surfaces were topically treated once with AAV-shRNA-Ifitm1 or AAV empty vector (control). Fourteen days after AAV application, corneas were subjected to NaOH injury. (9A) Significantly expressed in genes in stem/eTA cell cluster from our scRNA-seq data was subjected to AddModuleScore analysis, which uses a gene set that plays a role in regulating apoptosis from the MSigDB Collections. The overall expression score of genes related to apoptosis was calculated and indicated no difference in the expression of apoptosis-related genes in these 3 groups. n.s.: not significant. (9B) 24 hours after NaOH injury, immunofluorescent images didn't detect cleaved caspase 3 positive cells in the limbal epithelium of mice with AAV-shRNA-Ifitm1 or control. (N=4).

FIG. 10. Overexpression of Ifitm1 increases the expression of KRT15, a putative LESC marker. Mouse ocular surfaces were topically treated once with AAV-Ifitm1 cDNA (SEQ ID NO: 4) or AAV empty vector (control; AAV-EV). Fourteen days after AAV application, immunofluorescent staining showed IFITM1 and KERATIN 15 (KRT15) in the limbal epithelium of mice with AAV-Ifitm1 cDNA were increased compared to AAV-EV. N=10. Relative fluorescence intensity was analyzed using Image J. *p<0.05.

FIGS. 11A-11H. IFITM1 maintains an undifferentiated state of LESCs. (11A) HLECs, which are limbal epithelial stem/eTA cell-enriched, were transfected with siIFITM1 or siControl. Bulk RNA sequencing combined with gene ontology assay showed the top 50 differentially expressed genes (DEGs) were enriched in GO terms related to differentiation. (11B) We increased the calcium concentration to 1.6 mM (a calcium switch) in the culture medium to induce differentiation. 24 hours after a calcium switch, western blotting showed that knockdown of IFITM1 increased the expression of differentiation markers including mucin1 (Muc1), and plasminogen inhibitor-type 2 (PAI-2). Densitometry was conducted using ImageStudio. N=4. *: p<0.05. (11C) 24 hours after a calcium switch, RT-qPCR showed that knockdown of IFITM1 increased the expression of PAI-2, MUC1, and PIEZO1, while decreased putative limbal stem cell markers including GPHA2, ID3, and N-cadherin (N-cad) as well as Polycystin-2. N=4. *: p<0.05. (11D-11E) Mouse ocular surfaces were topically treated once with AAV-shRNA Ifitm1 or AAV-EV (control). 14 days after AAV application, immunostaining showed a decrease in p63 in the limbal epithelium of mice with AAV-shRNA Ifitm1 compared to AAV-EV (N=10). Relative fluorescence intensity was analyzed using Image J (11E). *p<0.05. (11F) Immunostaining with IFITM1 and Krt15 antibodies detects IFITM1 and Krt15 in limbal epithelial cells. IFITM1 is more highly expressed in the limbal epithelium than in the corneal epithelium (N=4). (11G) Violin plots show that the expression of PIEZO1 among epithelial clusters was not detected in the limbal stem/eTA cell cluster in WT mice. (11H) 24 hours after a calcium switch, RT-qPCR showed that knockdown of PIEZO11 decreased the expression of PIEZO1 as well as differentiation markers including PAI-2, MUC1, SPRR2D, SPRR2E, KRT12, and KRT3. N=4. *: p<0.05.

FIG. 12. Aging reduces IFITM1 expression while increases Ovol1 and Piezo1 in stem/eTA cells. scRNA-seq analysis of corneal/limbal cells between young (11 weeks old) vs aged mice (18 months old) was conducted. Violin plots showed that Ifitm1 expression was reduced while Ovol1 and Piezo1 were increased in aged corneas compared to young corneas.

FIG. 13. YAP localizes in corneal epithelial cell nuclei. Immunofluorescence staining using YAP and Krt15 antibodies reveals that YAP nuclear localization in corneal epithelial cells is higher than limbal epithelial cells in WT mice.

FIG. 14. Knockdown of PIEZO1 reduces F-actin. Immunofluorescence microscopy of HLECs stained with phalloidin (F-actin), and DAPI (nuclei) 72 hours after transfected with siPIEZO1. N=4. *: p<0.05.

### DETAILED DESCRIPTION

### Overview

In certain aspects, a pharmacological treatment for treating a corneal wound in a subject in need thereof is provided. In the same or alternative aspects, a pharmacological treatment for increasing a population of limbal epithelial stem cells (LESCs) and/or early transit amplifying cells (eTAs) in the limbal epithelium of a subject in need thereof is provided. The treatments can include a therapeutic agent that: i) results in an increase in concentration of IFITM1 (Interferon Induced transmembrane Protein 1) in the limbal epithelium of the subject; and/or ii) results in a decrease in concentration of OVOL1 (Ovo Like Zinc Finger 1) in the limbal epithelium of the subject. In another aspect, the disclosure provides an adeno-associated virus (AAV) comprising a heterologous nucleic acid sequence encoding IFITM1 or a variant thereof, operably linked to a regulatory sequence(s) which directs expression of the IFITM1 or the variant thereof.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a therapeutic agent” should be interpreted to mean “one or more therapeutic agents.” As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the term “modulate” means decreasing or inhibiting and/or increasing or augmenting. For example, modulating IFITM1 expression may mean increasing or augmenting IFITM1 expression and/or decreasing or inhibiting IFITM1 expression. Modulating IFITM1 biological activity may mean increasing or augmenting IFITM1 biological activity and/or decreasing or inhibiting IFITM1 biological activity. As another example, modulating OVOL1 expression may mean increasing or augmenting OVOL1 expression and/or decreasing or inhibiting OVOL1 expression. Modulating OVOL1 biological activity may mean increasing or augmenting OVOL1 biological activity and/or decreasing or inhibiting OVOL1 biological activity. The therapeutic agents disclosed herein may be administered to a subject in need thereof to modulate IFITM1 and/or OVOL1 expression and/or IFITM1 and/or OVOL1 biological activity.

### IFITM1

As used herein, the term “IFITM1” refers to the human protein encoded by the IFITM1 gene, or non-human equivalents thereof. IFITM1 may also be referred to as “Interferon Induced Transmembrane Protein 1.” Variants of IFITM1 are also contemplated herein. In various aspect, IFITM1 can have an amino acid sequence having at least about 85%, 90%, 95%, 96%, 97%, 98%, 99%, or 100% identity to SEQ ID NO: 1. In certain aspects a polynucleotide encoding for IFITM1 can have a nucleotide sequence having at least about 85%, 90%, 95%, 96%, 97%, 98%, 99%, or 100% identity to SEQ ID NO: 2.

### OVOL1

As used herein, the term “OVOL1” refers to the human protein encoded by the OVOL1 gene, or non-human equivalents thereof. OVOL1 may also be referred to as “Ovo like zinc finger 1,” or “Ovo like transcriptional repressor 1.” In various aspect, OVOL1 can have an amino acid sequence having at least about 85%, 90%, 95%, 96%, 97%, 98%, 99%, or 100% identity to SEQ ID NO: 5. In certain aspects a polynucleotide encoding for OVOL1 can have a nucleotide sequence having at least about 85%, 90%, 95%, 96%, 97%, 98%, 99%, or 100% identity to SEQ ID NO: 3.

### Subject in Need Thereof

As used herein, a “subject” may be interchangeable with “patient” or “individual” and means an animal, which may be a human or non-human animal, in need of treatment.

As used herein, the term “a subject in need thereof” refers to a human or non-human subject that can be treated with any of the therapeutic agents disclosed herein. A subject in need thereof may include a subject having a corneal wound or disease affecting the cornea, such as a subject having diabetes and exhibiting diabetic keratopathy.

### Therapeutic Agents

As used herein, a therapeutic agent may refer to any agent that is administering to a subject in need thereof in order to treat the subject. A therapeutic agent may refer to an agent that modulates expression and/or concentration of IFITM1 and/or OVOL1 in the limbal epithelium and/or the cornea of the subject. As an example, the agent can increase expression and/or concentration of IFITM1 and/or reduce the expression or concentration of OVOL1 in the limbal epithelium and/or the cornea of the subject. A therapeutic agent may refer to an agent that modulates a biological activity of IFITM1 and/or OVOL1 in the limbal epithelium and/or the cornea of the subject. Therapeutic agents may include, but are not limited to, small molecules or compounds, peptides, proteins (e.g., peptides or proteins comprising at least a fragment of the amino acid sequence of IFITM1 and/or OVOL1, and nucleic acids (e.g., nucleic acids encoding a peptide or protein comprising at least a fragment of the amino acid sequence of IFITM1 and/or OVOL1. Therapeutic agents may include, but are not limited to, pharmaceutical compositions comprising small molecules, compounds, peptides, proteins, and/or vectors (e.g., viral vectors) that express IFITM1 or variants and/or inhibit expression of OVOL1.

### Polynucleotides and Synthesis Methods

Polynucleotides and uses thereof may be disclosed herein such as polynucleotides encoding at least a fragment of the amino acid sequence of IFITM1 or a variant thereof, and/or at least a fragment of the amino acid sequence of OVOL1 or a variant thereof. The terms “polynucleotide (or “nucleic acid” and “oligonucleotide”) refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

A “fragment” of a polynucleotide is a portion of a polynucleotide sequence which is identical in sequence to but shorter in length than a reference sequence. As used herein, a reference sequence may include a polynucleotide sequence encoding IFITM1 and/or OVOL1 or variants thereof. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide; in other embodiments a fragment may comprise no more than 5, 10, 15, 20, 25, 30, 40, 50, 60, 70, 80, 90, 100, 150, 250, or 500 contiguous nucleotides of a reference polynucleotide; in further embodiments a fragment may comprise a range of contiguous nucleotides of a reference polynucleotide bounded by any of the foregoing values (e.g. a fragment comprising 20-50 contiguous nucleotides of a reference polynucleotide). Fragments may be preferentially selected from certain regions of a molecule. The term “at least a fragment” encompasses the full length polynucleotide. A “variant,” “mutant,” or “derivative” of a reference polynucleotide sequence may include a fragment of the reference polynucleotide sequence.

Regarding polynucleotide sequences and variants thereof, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “promoter” as used herein refers to a cis-acting DNA sequence that directs RNA polymerase and other trans-acting transcription factors to initiate RNA transcription from the DNA template that includes the cis-acting DNA sequence.

As used herein, the term “complementary” in reference to a first polynucleotide sequence and a second polynucleotide sequence means that the first polynucleotide sequence will base-pair exactly with the second polynucleotide sequence throughout a stretch of nucleotides without mismatch. The term “cognate” may in reference to a first polynucleotide sequence and a second polynucleotide sequence means that the first polynucleotide sequence will base-pair with the second polynucleotide sequence throughout a stretch of nucleotides but may include one or more mismatches within the stretch of nucleotides. As used herein, the term “complementary” may refer to the ability of a first polynucleotide to hybridize with a second polynucleotide due to base-pair interactions between the nucleotide pairs of the first polynucleotide and the second polynucleotide (e.g., A:T, A:U, C:G, G:C, G:U, T:A, U:A, and U:G).

As used herein, the term “complementarity” may refer to a sequence region on an anti-sense strand that is substantially complementary to a target sequence but not fully complementary to a target sequence. Where the anti-sense strand is not fully complementary to the target sequence, mismatches may be optionally present in the terminal regions of the anti-sense strand or elsewhere in the anti-sense strand. If mismatches are present, optionally the mismatches may be present in terminal region or regions of the anti-sense strand (e.g., within 6, 5, 4, 3, or 2 nucleotides of the 5′ and/or 3′ terminus of the anti-sense strand).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art.

The term “promoter” refers to a cis-acting DNA sequence that directs RNA polymerase and other trans-acting transcription factors to initiate RNA transcription from the DNA template that includes the cis-acting DNA sequence.

In certain exemplary embodiments, vectors such as, for example, expression vectors, containing a nucleic acid encoding one or more polypeptides and/or proteins described herein are provided (e.g., vectors encoding and/or expressing IFITM1 and/or OVOL1 or variants thereof). As used herein, the term “vector” refers to a nucleic acid molecule capable of transporting and/or expressing another nucleic acid to which it has been linked. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

As used herein, a “viral vector” (e.g., an adenovirus or adeno-associated virus, Sendai virus, or measles virus vector) refers to recombinant viral nucleic acid that has been engineered to express a heterologous polypeptide (e.g., IFITM1 and/or OVOL1 or a variant thereof) and/or to express a siRNA and/or shRNA for modulating OVOL1 and/or IFITM1 expression or activity. The recombinant viral nucleic acid typically includes cis-acting elements for expression of the heterologous polypeptide or nucleic acid element. The recombinant viral nucleic acid typically is capable of being packaged into a helper virus that is capable of infecting a host cell. For example, the recombinant viral nucleic acid may include cis-acting elements for packaging. Typically, the viral vector is not replication competent or is attenuated. An “attenuated recombinant virus” refers to a virus that has been genetically altered by modern molecular biological methods (e.g., restriction endonuclease and ligase treatment, and rendered less virulent than wild type), typically by deletion of specific genes. For example, the recombinant viral nucleic acid may lack a gene essential for the efficient production or essential for the production of infectious virus. The recombinant viral nucleic acid, packaged in a virus (e.g., a helper virus) may be introduced into a human subject by standard methods.

Numerous virus species can be used as the recombinant virus vectors for the pharmaceutical composition disclosed herein. A preferred recombinant virus is adeno-associated virus. Others include adenoviruses, retroviruses that are packaged in cells with amphotropic host range, vaccinia virus, canarypox, Sendai virus, measles virus, Yellow Fever vaccine virus (e.g., 17-D or similar), attenuated or defective DNA viruses, such as but not limited to herpes simplex virus (HSV), papillomavirus, and Epstein Barr virus (EBV).

In exemplary aspects, the recombinant expression vectors comprise a nucleic acid sequence in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif (1990).

In various aspects, the disclosure provides an adeno-associated viral (AAV) vector comprising a heterologous nucleic acid sequence encoding IFITM1 or a variant thereof, operably linked to a regulatory sequence(s) which directs expression of the variant thereof. In some embodiments, the IFITM1 or variant thereof has an amino acid sequence having at least about 85%, 90%, 95%, 96%, 97%, 98%, 99%, or 100% identity to SEQ ID NO: 1. In some embodiments, the AAV comprises a heterologous nucleic acid sequence having at least about 85%, 90%, 95%, 96%, 97%, 98%, 99%, or 100% identity to SEQ ID NO: 4.

In the same or alternative aspects, the AAV vector may comprise a heterologous nucleic acid sequence encoding an agent that can decrease the concentration of OVOL1 (e.g., in the cornea and/or limbal epithelium) as compared to a concentration of OVOL1 (e.g., in the cornea and/or limbal epithelium) in the subject prior to administering the therapeutic agent. In certain aspects, the agent encoded by the heterologous nucleic acid sequence may comprise one or more siRNAs or shRNAs capable of decreasing OVOL1 expression.

As used herein, the term “adeno-associated virus” (AAV) includes, without limitation, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388), which are also encompassed by the term “AAV.” The genomic sequences of various AAV and autonomous parvoviruses, as well as the sequences of the ITRs, Rep proteins, and capsid subunits are known in the art. Such sequences may be found in the literature or in public databases such as the GenBank database.

### Peptides, Polypeptides, Proteins, and Synthesis Methods

Peptides, polypeptides, and proteins and uses thereof may be disclosed herein such as peptides, polypeptides, and proteins comprising at least a fragment of the amino acid sequence of IFITM1 and/or OVOL1 or a variant thereof. As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, 0-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110).

Reference may be made herein to peptides, polypeptides, proteins and variants thereof. Reference amino acid sequences may include, but are not limited to, an amino acid sequence comprising at least a fragment of the amino acid sequence of IFITM1 and/or OVOL1 or a variant thereof. Variants as contemplated herein may have an amino acid sequence that includes conservative amino acid substitutions relative to a reference amino acid sequence. For example, a variant peptide, polypeptide, or protein as contemplated herein may include conservative amino acid substitutions and/or non-conservative amino acid substitutions relative to a reference peptide, polypeptide, or protein. “Conservative amino acid substitutions” are those substitutions that are predicted to interfere least with the properties of the reference peptide, polypeptide, or protein, and “non-conservative amino acid substitution” are those substitution that are predicted to interfere most with the properties of the reference peptide, polypeptide, or protein. In other words, conservative amino acid substitutions substantially conserve the structure and the function of the reference peptide, polypeptide, or protein. The following table provides a list of exemplary conservative amino acid substitutions.

TABLE 1

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally disrupt: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain.

Variants comprising deletions relative to a reference amino acid sequence of peptide, polypeptide, or protein are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

Variants comprising fragment of a reference amino acid sequence of a peptide, polypeptide, or protein are contemplated herein. A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide.

Variants comprising insertions or additions relative to a reference amino acid sequence of a peptide, polypeptide, or protein are contemplated herein. The words “insertion” and “addition” refer to changes in an amino acid or sequence resulting in the addition of one or more amino acid residues.

Fusion proteins also are contemplated herein. A “fusion protein” refers to a protein formed by the fusion of at least one peptide, polypeptide, or protein or variant thereof as disclosed herein to at least one heterologous protein peptide, polypeptide, or protein (or fragment or variant thereof). The heterologous protein(s) may be fused at the N-terminus, the C-terminus, or both termini of the peptides or variants thereof.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide (e.g., glycosylase activity or other activity). “Substantially isolated or purified” amino acid sequences are contemplated herein. The term “substantially isolated or purified” refers to amino acid sequences that are removed from their natural environment, and are at least 60% free, preferably at least 75% free, and more preferably at least 90% free, even more preferably at least 95% free from other components with which they are naturally associated.

### Pharmaceutical Formulation

The disclosed therapeutic agents may be formulated as pharmaceutical compositions for administering to a subject in need thereof. The disclosed pharmaceutical compositions may include: (a) a therapeutic agent as discussed herein (e.g., an effective amount of a therapeutic agent for treating corneal wounds); and (b) one or more pharmaceutically acceptable carriers, excipients, or diluents.

The therapeutic agents utilized in the methods disclosed herein may be formulated as a pharmaceutical composition that includes one or more carrier agents, binding agents, filling agents, lubricating agents, suspending agents, buffers, wetting agents, and/or preservatives. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The therapeutic agents utilized in the methods disclosed herein may be formulated as a pharmaceutical composition for delivery via any suitable route. In one aspect, for example, the pharmaceutical composition may be administered to the limbal epithelium, conjunctiva, or cornea of the subject, e.g., topically.

The therapeutic agents utilized in the methods disclosed herein may be administered in conventional dosage forms prepared by combining the active ingredient with standard pharmaceutical carriers or diluents according to conventional procedures well known in the art. These procedures may involve mixing or dissolving the ingredients as appropriate to the desired preparation.

Pharmaceutical compositions comprising the therapeutic agents may be adapted for administration by any appropriate route, for example topically to the surface of the cornea, limbal epithelium, or conjunctiva. Such formulations may be prepared by any method known in the art of pharmacy, for example by bringing into association the active ingredient with the carrier(s) or excipient(s).

In various aspects, the therapeutic agents can include any agent that can increase the concentration of IFITM1 as compared to a concentration of IFITM1 in the subject prior to administering the therapeutic agent. In the same or alternative aspects, the therapeutic agents can include any agent that can increase the concentration of IFITM1 in the cornea and/or limbal epithelium of the subject as compared to a concentration of IFITM1 in the cornea and/or limbal epithelium of the subject prior to administering the therapeutic agent. In the same or alternative aspects, administering the therapeutic agents can increase activation of stem cells and/or expansion of eTA cell population in the cornea and/or limbal epithelium of the subject, as compared to the stem cell activation and/or eTA cell population prior to administering the therapeutic agent. In various aspects, the therapeutic agents can result in faster or more complete healing of the corneal wound, as compared to the healing of a corneal wound in a subject who has not been administered the therapeutic agents. In certain aspects, the therapeutic agent is a vector that expresses IFITM1 or variant thereof. In one or more aspects, the vector can be a viral vector. In various aspects, the viral vector can be an adenovirus-associated viral vector that expresses IFITM1 or variant thereof. In certain aspects, the therapeutic agent can be the IFITM1 protein or variant thereof. In various aspects, the IFITM1 protein or variant thereof can be fused to one or more cell penetrating peptides or other cell penetrating agents. In one or more aspects, the IFITM1 protein can be a component of a fusion protein.

In the same or alternative aspects, the therapeutic agent can include any agent that can decrease the concentration of OVOL1 as compared to a concentration of OVOL1 in the subject prior to administering the therapeutic agent. In various aspects, the therapeutic agents can include any agent that can decrease the concentration of OVOL1 in the cornea and/or limbal epithelium of the subject as compared to a concentration of OVOL1 in the cornea and/or limbal epithelium of the subject prior to administering the therapeutic agent. In certain aspects, the therapeutic agent can comprise one or more siRNAs or shRNAs capable of decreasing OVOL1 expression. In various aspects, the siRNA and/or shRNA can be present on a vector, such as one or more of the viral vectors disclosed herein.

### Methods

In various aspects, methods are disclosed for treating a corneal wound and/or increasing and/or activating a population of limbal epithelial stem cells (LESCs) and/or early transit amplifying cells (eTAs) in the limbal epithelium of a subject. In various aspects, the subject can exhibit a corneal wound that is caused at least partly by a physical wound, chemical burn, or disease. In one aspect, such a disease can include diabetes and the subject can exhibit diabetic keratopathy.

In one or more aspects, the methods can include administering one or more therapeutic agents disclosed herein. In various aspects, the methods can include administering one or more therapeutic agents disclosed herein that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decreased concentration of OVOL1 (Ovo Like Zinc Finger 1) in the limbal epithelium of the subject compared to the concentration of OVOL1 in the limbal epithelium of the subject prior to administering the therapeutic agent. In the same or alternative aspects, administering the therapeutic agent can increase activation of stem cells and/or expansion of eTA cell population in the cornea and/or limbal epithelium of the subject, as compared to the stem cell activation and/or eTA cell population prior to administering the therapeutic agent. In various aspects, administering the therapeutic agent can result in faster or more complete healing of the corneal wound, as compared to the healing of a corneal wound in a subject who has not been administered the therapeutic agent.

In various aspects, the therapeutic agent can be administered to the limbal epithelium, conjunctiva, or cornea of the subject. In such aspects, any convenient administration procedures can be used to the deliver the therapeutic agent to the limbal epithelium, conjunctiva, or cornea of the subject.

### EXAMPLES

The following examples are illustrative and should be interpreted to limit the scope of the claimed subject matter.

### Example 1—IFITM1/OVOL1 Axis is a Novel Regulator of Limbal Epithelial Stem Cell Activation and eTA Cell Expansion

Limbal epithelial stem cells (LESCs) reside in the basal layer of the limbal epithelium. Under normal conditions, LESCs rarely proliferate. Upon wounding, these stem cells are activated to repair the corneal epithelium, a process involved in maintaining corneal epithelial homeostasis and tissue transparency, which is essential for clear vision. Stem cell activation and consequent eTA expansion are one of the strategies by which LESCs respond to corneal epithelial wounding. To understand how these processes are regulated, we conducted a single cell RNA sequencing assay and found that IFITM1 (Interferon Induced Transmembrane Protein 1) is predominantly expressed in stem/early transit amplifying (stem/eTA) cells and positively associated with stem/eTA cell population expansion after corneal wounding, indicating a positive role of IFITM1 in stem/eTA cell population expansion. To strongly support this idea, knockdown of IFITM1 using an AAV (adeno-associated virus) vector significantly attenuated the stem/eTA cell expansion after corneal wounding in vivo. In vitro studies suggest that the positive role of IFITM1 in activation of the proliferation of stem/eTA cell-enriched limbal epithelial cells contributes to expansion of stem/eTA cell population, at least partially, via inhibiting OVOL1(Ovo like zinc finger 1), a negative regulator of epithelial cell proliferation. These results provide a rationale for developing a novel therapy utilizing AAV to deliver and overexpress IFITM1, aimed at treating not only corneal diseases caused by wounding but also those associated with compromised LESCs.

### Introduction

The corneal epithelium is a self-renewing stratified squamous epithelium¹⁻⁶. A unique feature of the corneal epithelium is that the stem cells, which govern self-renewing tissues, are not located in the corneal epithelium; rather they are situated in the basal layer of the limbal epithelium (called limbal epithelial stem cells (LESCs); see^{1, 4, 5, 7-10} and references therein). LESCs possess stem cell properties (“stemness”) such as: quiescence, extensive proliferative capacity, expression of putative stem cell markers (e.g., Gpha2), and can generate committed progeny: transit amplifying (TA) cells^{1-4, 5, 7-16}. Early TA cells (eTA), which represent immediate stem cell progeny, also reside in the limbal epithelial basal layer and have high proliferative capacity^{3, 5}. It is believed that LESCs and eTA cells are phenotypically and functionally indistinguishable^{17, 18}. LESCs are involved in maintaining corneal epithelial homeostasis. Loss or dysfunction of LESCs results in persistent epithelial defects and loss of vision^{19-26, 28, 29}. Therefore, it is clinically important to understand the molecular signaling pathways that are involved in regulating stem cell biology including stem cell proliferation.

In addition to corneal epithelial homeostasis, LESCs also play a role in corneal wound healing. In response to corneal injury including chemical burn or the physical wounding of the central corneal epithelium, limbal basal layers produces more corneal epithelial cells, which is essential for re-epithelialization^{3, 27, 28}. For decades, it has been demonstrated that one of the strategies to produce more corneal epithelial cells is to activate LESCs to proliferate, thus expanding stem/eTA cell population³. Since the expansion of stem/eTA cell population is involved in corneal wound healing, understanding the molecular mechanisms how such expansion is regulated is valuable.

IFITM1 (Interferon Induced Transmembrane Protein 1) is an interferon induced antiviral proteins. Recently, IFITM1 drew more attention since accumulating evidence shows it exerts other functions, such as its involvement in maintenance of stem cells. IFITM1 is highly expressed in human embryonic stems cells and that the expression progressively decreases when stem cells differentiate²⁹. IFITM1 is also expressed in mouse primordial germ cells (PGCs) indicating a role in PGC development³⁰. In addition to stem cells, IFITM1 plays a role in cancer stem cells. For example, IFITM1 is involved in the progression of nonsmall cell lung cancer (NSCLC) in patients³¹. Loss of IFITM1 leads to a significant reduction in sphere formation, which is considered a unique feature of cancer stem cells³¹. In colorectal cancer, IFITM1^{high} cells form organoids with more proliferating cells³². Such positive role of IFITM1 in cancer cell proliferation is also detected in cervical squamous cell carcinoma³³. Overall, these suggest that IFITM1 has a positive role in proliferation in cancer cells.

OVOL1/2 (Ovo like zinc finger 1/2), encoding zinc finger protein homologous to Drosophila melanogaster Ovo, are involved in development of various tissues including cornea and epidermis³⁴. OVOL2 plays a role in maintaining the transcriptional profile of corneal epithelial cells by repressing mesenchymal genes³⁵. Mutations in OVOL2 have been associated with posterior polymorphous corneal dystrophy, an inherited disorder of the corneal endothelium³⁶. Loss of OVOL1 leads to a hyperproliferative defect in epidermis during development³⁷, indicating an inhibitory role of OVOL1 in cell proliferation. Consistently, OVOL1 is downregulated in cutaneous squamous cell carcinoma³⁸ and mediates the suppression of proliferation in oral squamous cell carcinoma cells by inhibiting ZEB1(Zinc Finger E-Box Binding Homeobox 1) expression³⁹. However, unlike OVOL2, the role of OVOL1 in limbus/cornea remains unclear.

Here our single cell RNA sequencing (scRNA seq) data identified several novel regulators that may be responsible for expanding stem/eTA cell population after corneal injury. Among them, we found that IFITM1 was upregulated in stem/eTA cell population after corneal injury. AAV (adeno-associated virus) delivered shRNA-Ifitm1 reduced Ifitm1 expression in limbal epithelium and attenuated the expansion of stem/eTA cell population in response to corneal injury. Our findings provide a rational for developing a novel therapy based on AAV delivering and overexpressing IFITM1 to treat the corneal diseases with the limbal stem cell dysfunction such as diabetic keratopathy.

### Results

scRNA-Seq Analyses Reveal that Corneal Injury with NaOH Results in Expansion of Stem/eTA Cell Population

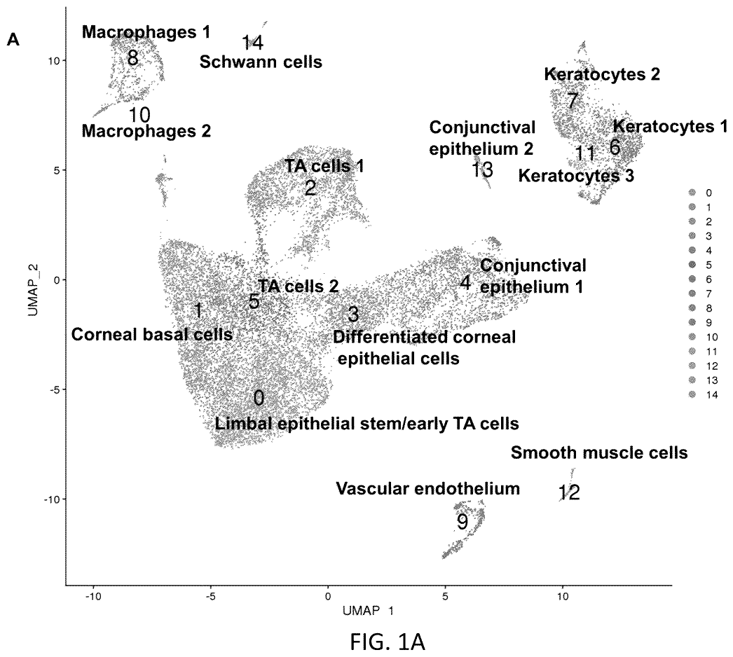

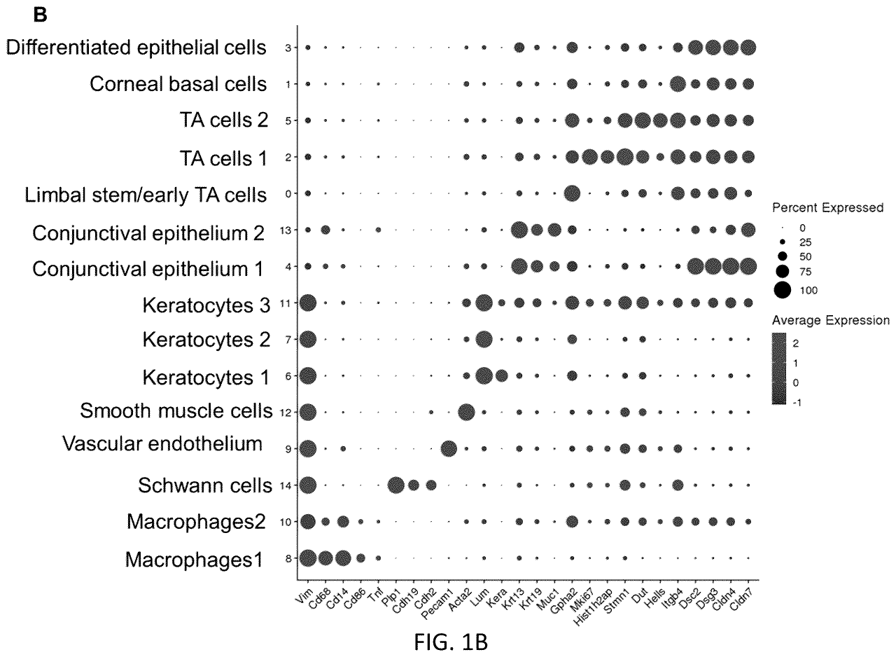

To understand how expanding stem/eTA cell population is regulated after corneal injury, scRNA-seq analysis using cells from mouse corneas that were exposed to 1M NaOH solution for 30s as well as uninjured controls. scRNA-seq data were analyzed using Seurat and 15 distinct clusters were identified and visualized in a UMAP plot (FIG. 1A). In these 15 clusters, the percentage of the cells expressing marker genes and average expression levels of marker genes were visualized using a dot plot (FIG. 1). Among the 15 clusters, 8 cell clusters were mesenchymal cells, which expressed the mesenchymal marker Vimentin (Vim). These 8 clusters included 3 keratocyte clusters, corneal Schwann cell cluster, smooth muscle cell cluster, vascular endothelial cell cluster, and 2 macrophage clusters.

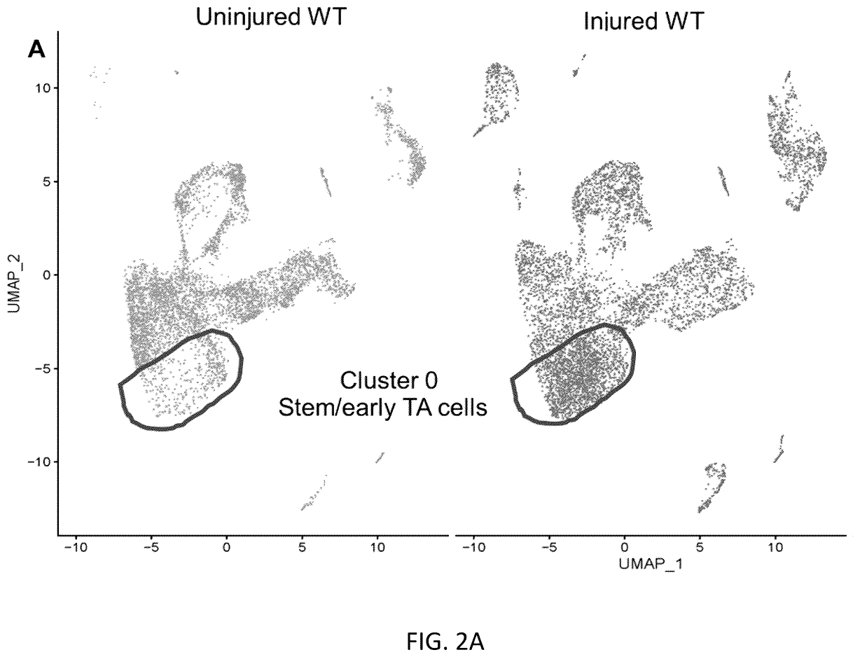

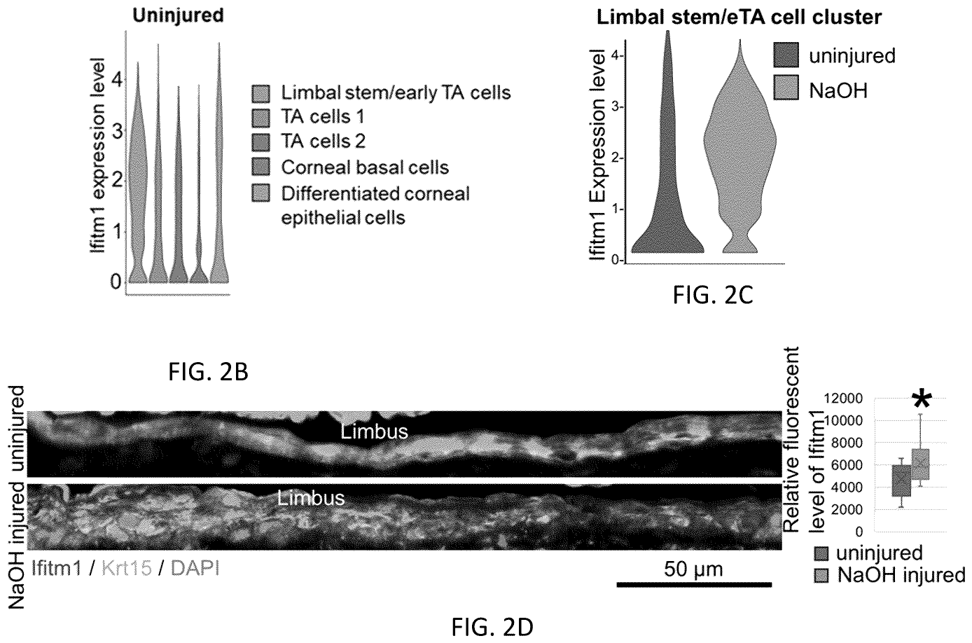

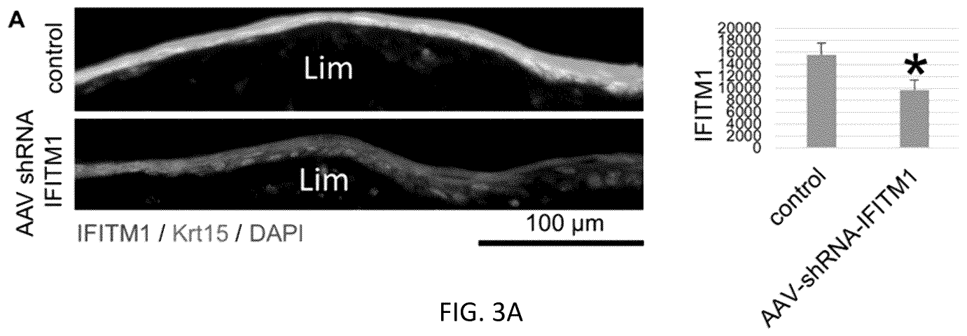

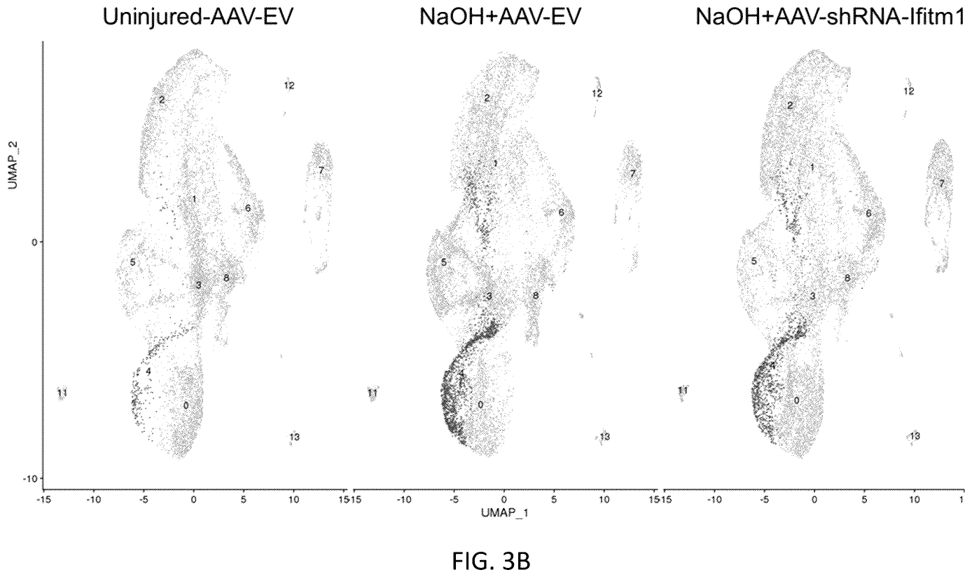

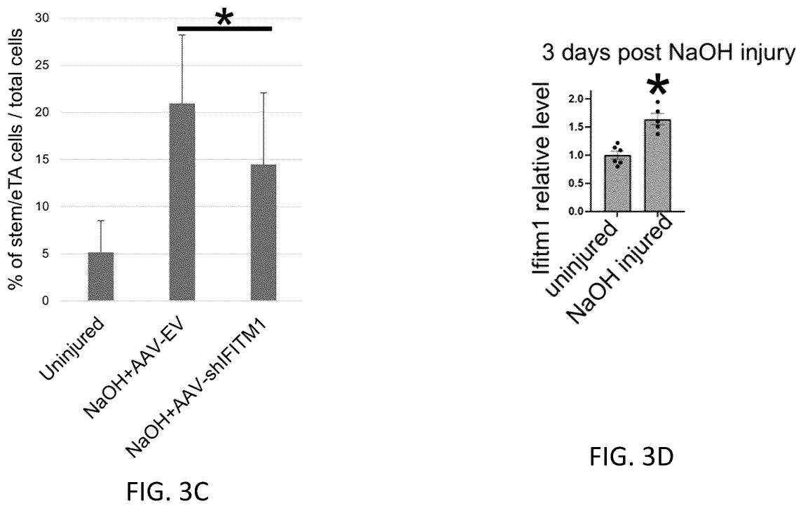

The remaining seven cell clusters were identified as epithelial in nature. Cluster 4 and 13 were identified as conjunctival epithelial cells based on high expression of conjunctival markers such as keratin 13 (Krt13), keratin 19 (Krt19) and mucin1 (Muc1)^{15,40,41} (FIG. 1). Cluster 0 highly expressed Glycoprotein Hormone Subunit Alpha 2(Gpha2), a limbal epithelial stem/eTA cell marker^{15,16}. In addition, cluster 0 expressed low levels of differentiation markers such as desmocollin-3 (Dsc3) and desmoglein 3 (Dsg3)^{15,42}. This suggests that cluster 0 is predominately limbal epithelial stem/eTA cells. Clusters 2 and 5 were identified as more mature TA cell populations since TA cell markers including the marker of proliferation Ki-67 (Mki67), histone cluster 1, H2ap (Hist1h2ap), stathmin 1 (Stmn1), deoxyuridine triphosphatase (Dut), and helicase, lymphoid specific (Hells)^{18,43-45} were enriched in these two clusters. Cluster 1 highly expressed basal cell markers (e.g., Integrin beta 4 (Itgb4))^{46, 47}; while cluster 3 highly expressed differentiation markers (e.g., desmosome proteins, tight junction proteins)^{15,42}. Thus, cluster 1 was designated as corneal epithelial basal cells and cluster 3 as differentiated corneal epithelial cells. Consistent with the idea that stem cells are activated to generate more eTA cells in response to, scRNA-seq demonstrated that the percentage of stem/eTA cells increased in the NaOH injured WT cornea by an average 20% compared to uninjured WT cornea (average 5%) (FIGS. 2A and 3C) indicating an activation of stem cells and an expansion of eTA cell population.

scRNA-Seq Analyses Identify that Ifitm1 is a Novel Regulator for Expansion of Stem/Early TA Cells in Response to Corneal Injury

Activation of quiescent stem/eTA cells to proliferate leads to expanding stem/eTA cell population³. To understand how the expansion of stem/early TA cells is controlled, we searched for genes related to proliferation among the top of the differentially expressed genes (DEGs) in stem/early TA cells (cluster 0) between uninjured and injured. IFITM1 was among the most highly differentially expressed genes (DEGs) in the stem/eTA cell cluster between uninjured and injured corneas. IFITM1 was predominantly expressed in the stem/eTA cell cluster in uninjured mouse corneas (FIG. 2B) as well as human corneas. Bulk RNA seq suggested that IFITM1 is significantly up-regulated in label retaining cells in mouse limbal epithelium⁴ and colocalized with C/EBPδ, a putative stem cell marker, in human limbal basal layer⁴⁹. It has been shown that IFITM1 has a positive role in proliferation in cancer cells^{32,33}. Thus, Ifitm1 may play a role in regulating proliferation of limbal epithelial stem/eTA cells. Following corneal injury, Ifitm1 expression was upregulated in stem/early TA cell population compared with uninjured control (FIG. 2C). To validate this, immunostaining detected an increase in Ifitm1 expression in limbal epithelium of NaOH injured mouse corneas compared with uninjured controls (FIG. 2D). Not surprisingly, RT-qPCR and immunostaining showed that IFITM1 expression was markedly upregulated in mouse corneas 3 days after a NaOH burn (FIGS. 2D and 3D).

### IFITM1 is Involved in the Activation of Stem Cells and Expansion of the eTA Cell Population in Response to Corneal Injury

To examine whether the upregulation of Ifitm1 contributes to the expansion of stem/early TA cells in response to corneal injury, wild type mouse eyes were topically treated with AAV-shRNA-Ifitm1 or AAV-empty vector (control) and subsequently were subjected to corneal NaOH burn. AAV-shRNA-Ifitm1 treatment downregulated Ifitm1 expression in mouse limbal epithelium (FIG. 3A). A scRNA-seq assay was conducted using cells isolated from corneal/limbal tissues at three days after corneas were injured. scRNA-seq assay identified stem/early TA cell population using Gpha2, a limbal epithelial stem/eTA cell marker^{15,16}. In AAV-EV treated mouse eyes, NaOH injury increased the numbers of the stem/eTA cells compared to uninjured eyes. Such expansion of stem/eTA cell population following corneal injury was markedly reduced in AAV-shRNA-Ifitm1 treated eyes (FIG. 3B). This suggests a positive role of Ifitm1 in stem/early TA cell expansion after corneal injury.

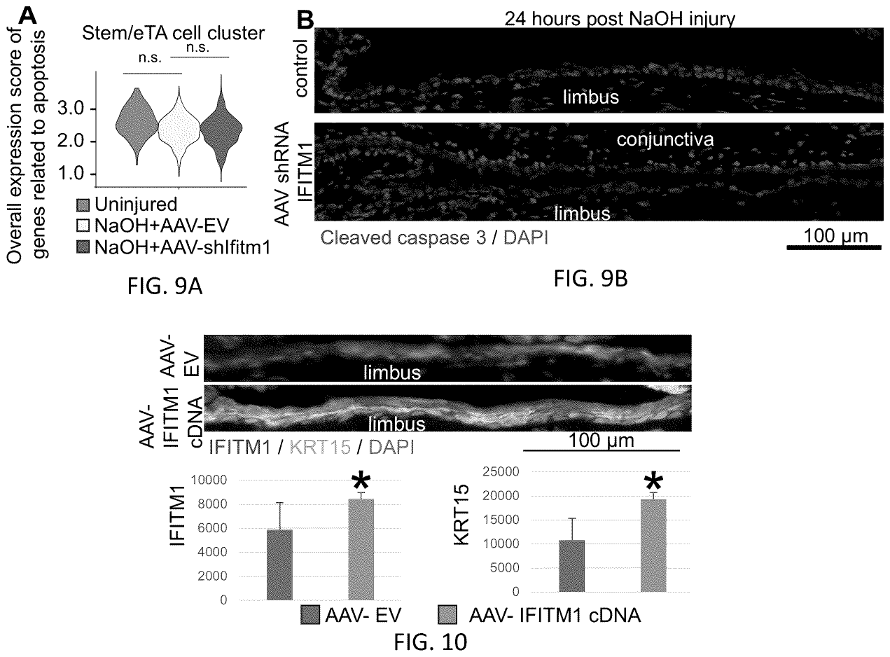

AddModuleScore analysis⁶³ found that there was no significant difference in the overall activity of the gene set related to apoptosis in the stem/eTA cell cluster of uninjured, NaOH+AAV-EV and NaOH+AAV-shIfitm1-treated mice (FIG. 9A). Consistently, immunostaining for cleaved caspase 3 did not detect any positive cells in the limbal epithelium of both NaOH+AAV-EV and NaOH+AAV-shIfitm1-treated mice (FIG. 9B). These observations provide evidence that the effect of depletion of Ifitm1 is not due to alteration of cell viability.

### IFITM1 Positively Regulates Proliferation in Limbal Epithelial Cells In Vitro

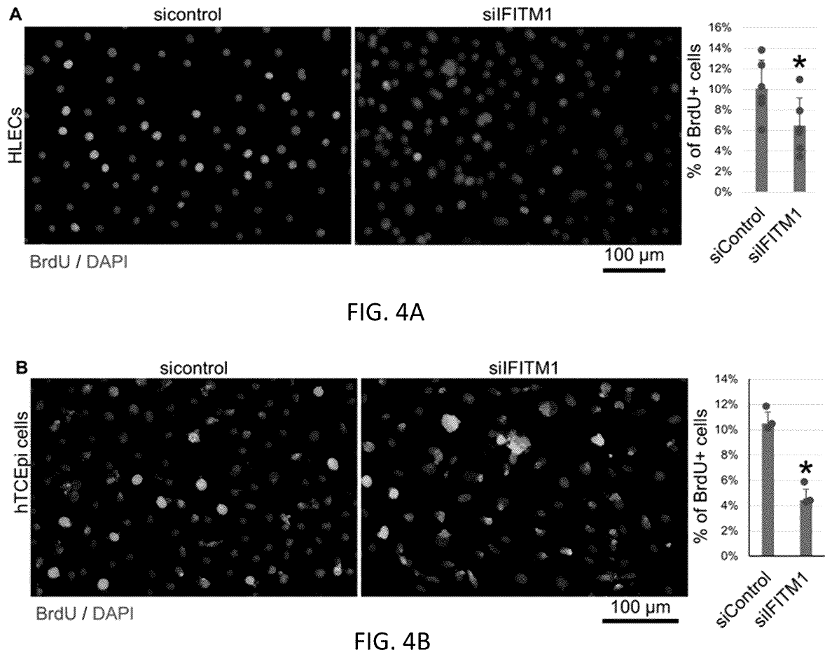

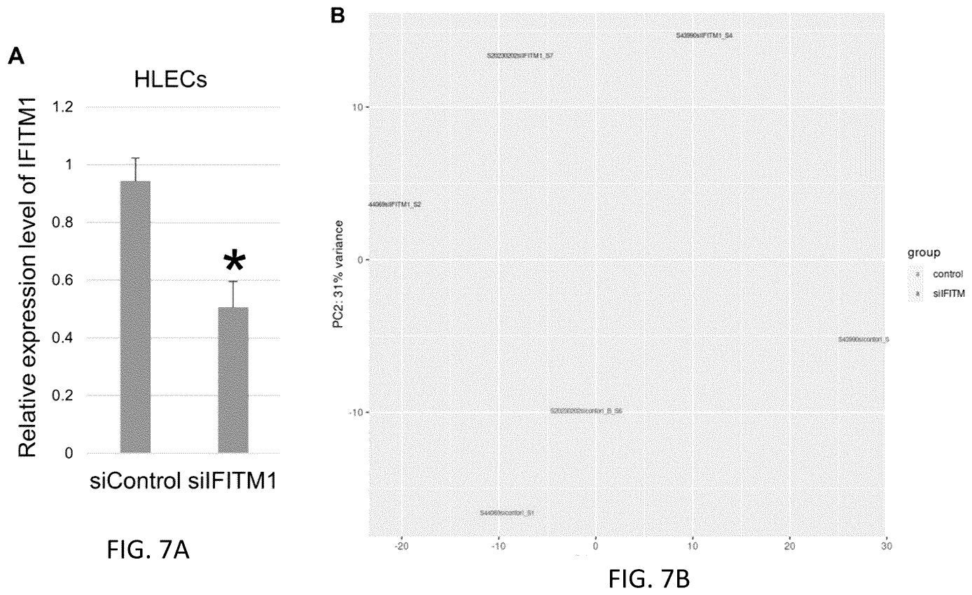

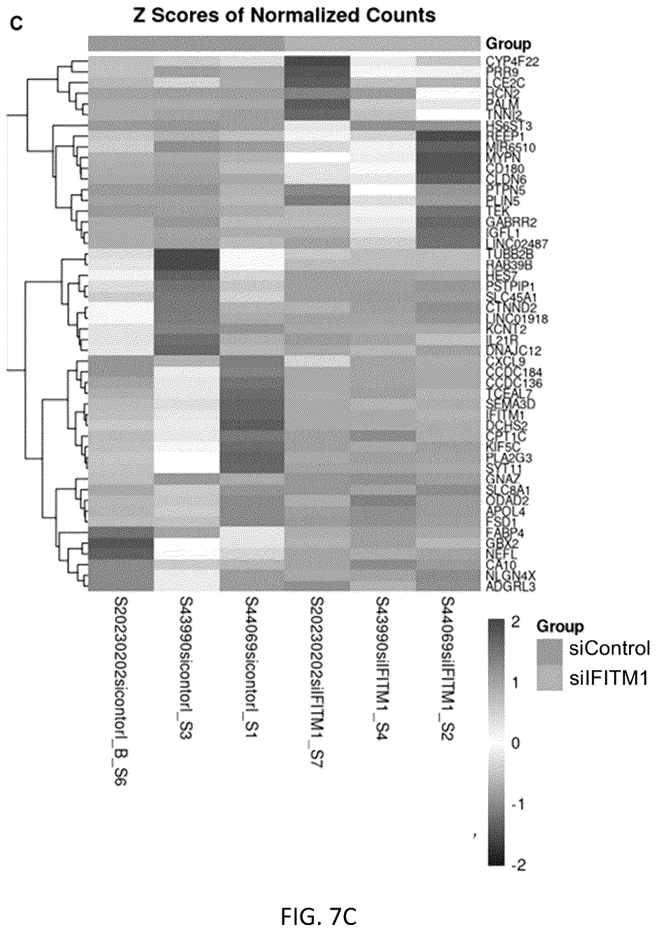

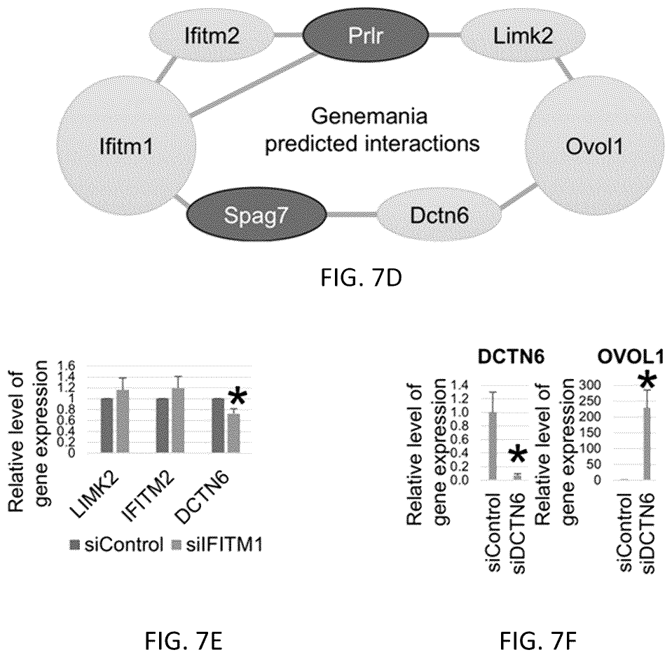

Following corneal injury, LESCs are activated to proliferate thus expanding stem/eTA cell population⁶⁴. To investigate whether the positive role of IFITM1 in the expansion of stem/eTA cells is due to the positive role of IFITM1 in the proliferation, we utilized stem cell-enriched primary culture of human limbal epithelial cells (HLECs). These cells were transfected with siRNA pools against IFITM1 (siIFITM1) and a significant reduction of IFITM1 in these cells was detected (FIG. 7A). To assess cell proliferation, a BrdU labeling assay was conducted, which detects cells in the S phase of DNA synthesis. After a 72-hour transfection period, we observed a marked increase in the percentage of BrdU positive cells in siIFITM1 transfected cells when compared to the scrambled control siRNA (siControl) transfected cells (FIG. 4A). Such observation is also confirmed in hTCEpi cells, a human limbal epithelial cell line, evidenced by reduction of BrdU positive cells in siIFITM1 transfected hTCEpi cells (FIG. 4B). This suggests that IFITM1 has a positive role in regulation of stem cell-enriched limbal epithelial proliferation in vitro.

### IFITM1 Positively Regulates Proliferation in Limbal Epithelial Cells In Vivo

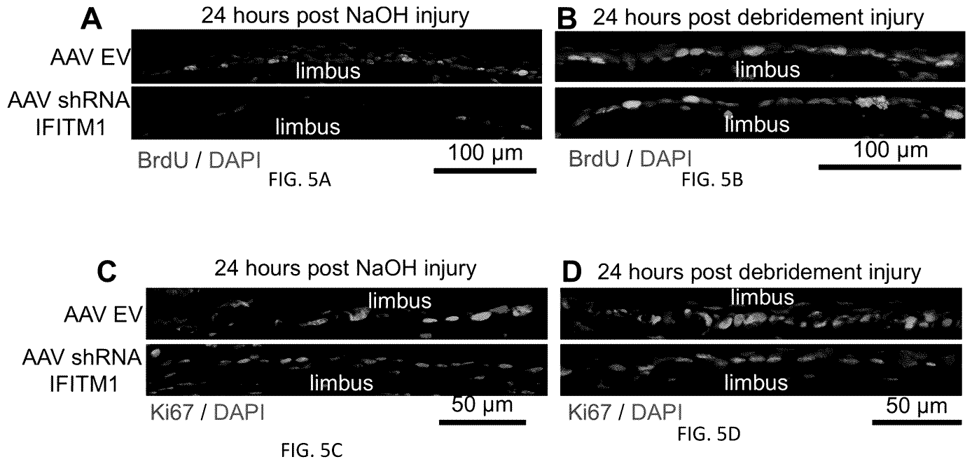

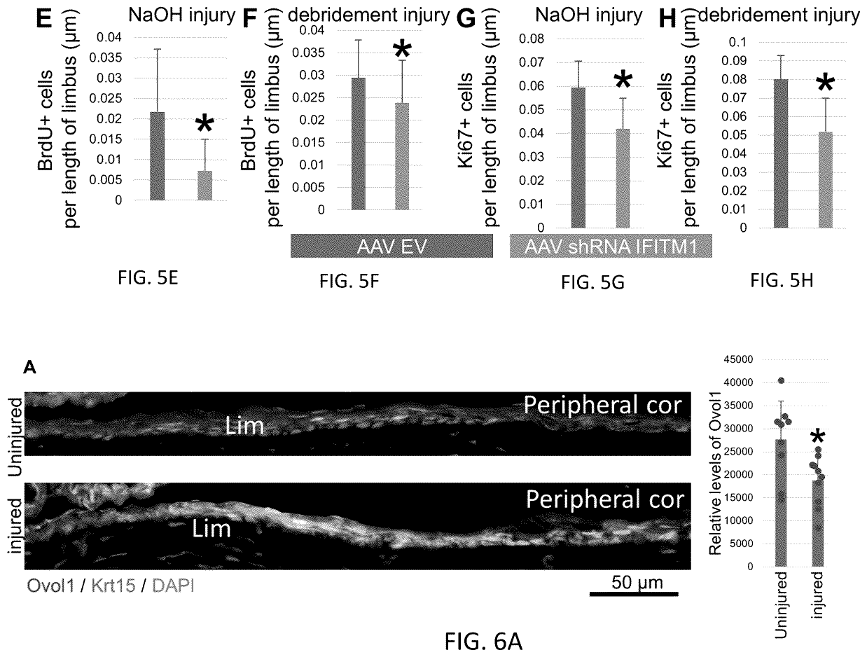

In response to corneal injury, expanding stem/early TA cell population results in increased proliferative cells in limbal epithelium 22. Thus, to determine the effect of Ifitm1 in the activation of the proliferation of LESCs in vivo, we investigate whether knockdown of Ifitm1 reduces proliferation in limbal epithelium after injury. Wild type mouse eyes were topically treated with AAV-shRNA-Ifitm1 or AAV-EV (control) and subsequently were subjected to either corneal epithelial debridement wounding or NaOH burn. We conducted a BrdU labeling assay, as well as immunostaining for Ki67, a well-established marker for cell proliferation. Twenty-four hours after corneal injury, knockdown of Ifitm1 by AAV-shRNA-Ifitm1 diminished BrdU+ cells and Ki67+ cells in limbal epithelium compared with control (FIGS. 5A-5H). This suggests that Ifitm1 is involved in the proliferation of limbal epithelium in response to corneal injury in vivo, indicating a role of Ifitm1 in stem cell activation and eTA cell expansion following injury.

### IFITM1 Positively Regulates Proliferation Via Negatively Regulating OVOL1

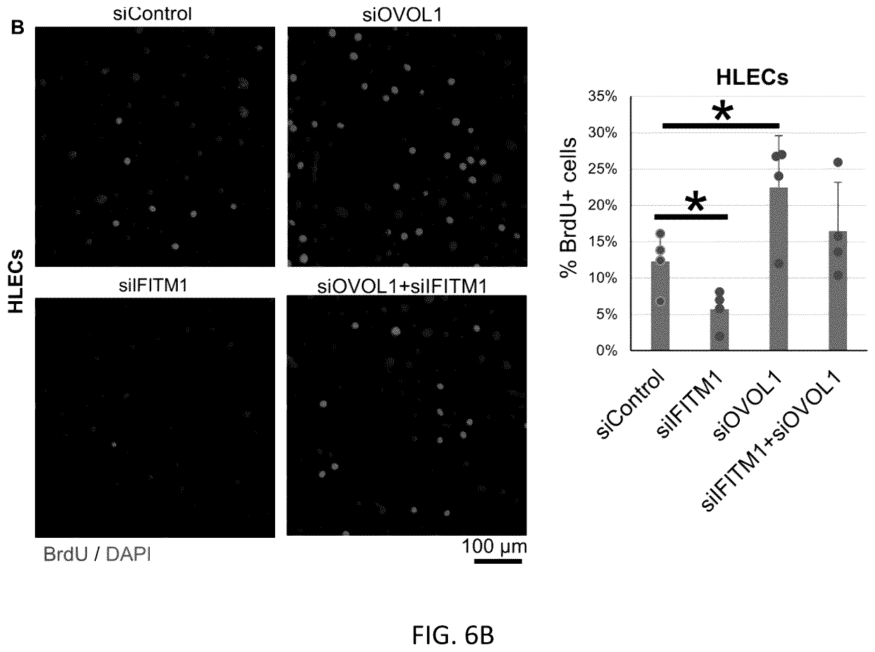

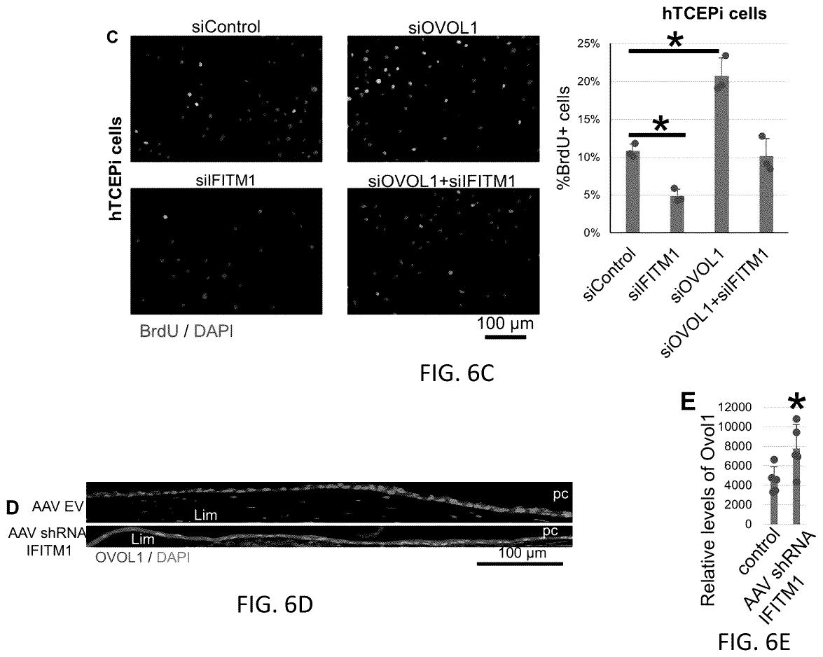

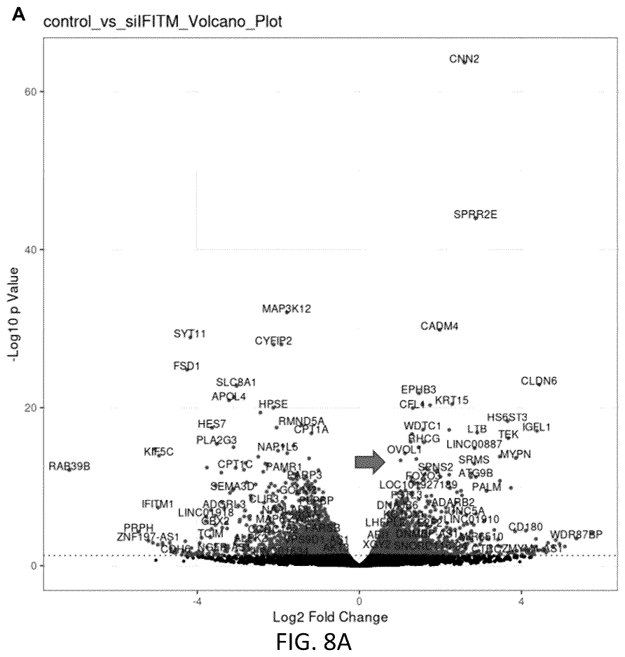

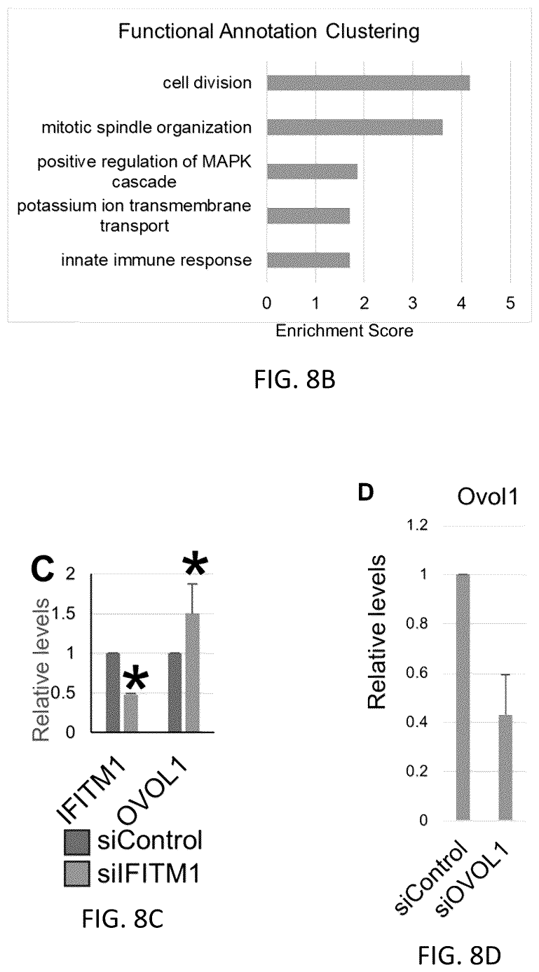

To explore how IFITM1 positively regulates limbal epithelial cell proliferation, an RNA sequencing assay was conducted using RNAs isolated from HLECs transfected with siIFITM1 or siControl (FIG. 7). RNA sequencing identified that 945 significantly, differentially expressed genes (DEGs) showed more than 2 fold changes between siIFITM1 and siControl treated HLECs (FIG. 8A). These DEGs were subjected to a gene ontology (GO) analysis (FIG. 8B). The top one of the significant GO terms was “cell division”. Among these genes involving “cell division”, Ovol1 plays a role in regulating proliferation and cell fate commitment in epidermal keratinocytes³⁷. Our RNA sequencing and RT-qPCR showed that knockdown of IFITM1 significantly increased OVOL1 expression (FIG. 8C). Interestingly, NaOH burn in mouse corneas reduced Ovol1 (FIG. 6A) in limbal epithelium compared to uninjured mice, while increased Ifitm1 expression (FIG. 2D). This in vivo observation confirmed the negative regulation of OVOL1 expression by IFITM1. Thus, to explore whether the decreased proliferation in cells with knockdown of IFITM1 is due to increased OVOL1 expression, we conducted a rescue experiment by knocking down OVOL1 in cells lacking IFITM1. RT-qPCR results confirmed a significant decrease of OVOL1 or IFITM1 expression after cells transfected with siRNA pools against OVOL1(siOVOL1) or siRNA pools against IFITM1 (siIFITM1), respectively (FIGS. 6B-6C). Knockdown of OVOL1 in both HLECs and hTCEpi cells resulted in a marked increase in the percentage of BrdU+ cells (FIG. 6D). Knockdown of IFITM1 reduced the percentage of BrdU+ cells while such reduction was reversed by knockdown of OVOL1. This indicates that IFITM1 positively regulates cell proliferation, at least partially, via negatively regulating OVOL1. In mice, topical treatment of AAV-shRNA-Ifitm1 markedly enhanced OVOL1 expression in the limbal epithelium compared to AAV-EV control (FIGS. 6D-6E), indicating IFITM1 negatively regulates OVOL1 expression in vivo.

### IFITM1 Regulates OVOL1 Via DCTN6

To explore how IFITM1 regulates OVOL1, we conducted a network analysis using GeneMANIA⁶⁵, which predicted a regulatory network that connects IFITM1 with OVOL1. In this predicted network, our bulk RNA sequencing indicated that the expressions of LIMK2, IFITM2, and DCTN6 were altered by knocking down IFITM1(GSE278535)⁶⁶. Among the 3 DEGs (LIMK2, IFITM2, and DCTN6), RT-qPCR only detected a significant reduction of DCTN6 expression in HLECs cells transfected with siIFITM1 compared with siControl (FIG. 4G). RT-qPCR showed that the knockdown of DCTN6 markedly upregulated OVOL1 expression in HLECs (FIG. 4H). These observations suggest that IFITM1 negatively regulates OVOL1 expression by positively modulating DCTN6 expression.

### Overexpression of Ifitm1 Increases the Expression of KRT15, a Putative LESC Marker

We produced an AAV vector to express Ifitm1 cDNA in mouse limbal/corneal epithelia. Topical application of AAV-Ifitm1-cDNA on wild type mouse cornea increased the expression of IFITM1 and KRT15 in limbal epithelium (FIG. 10).

Downregulation of Iftm1 is Associated with Dysfunction of LESCs

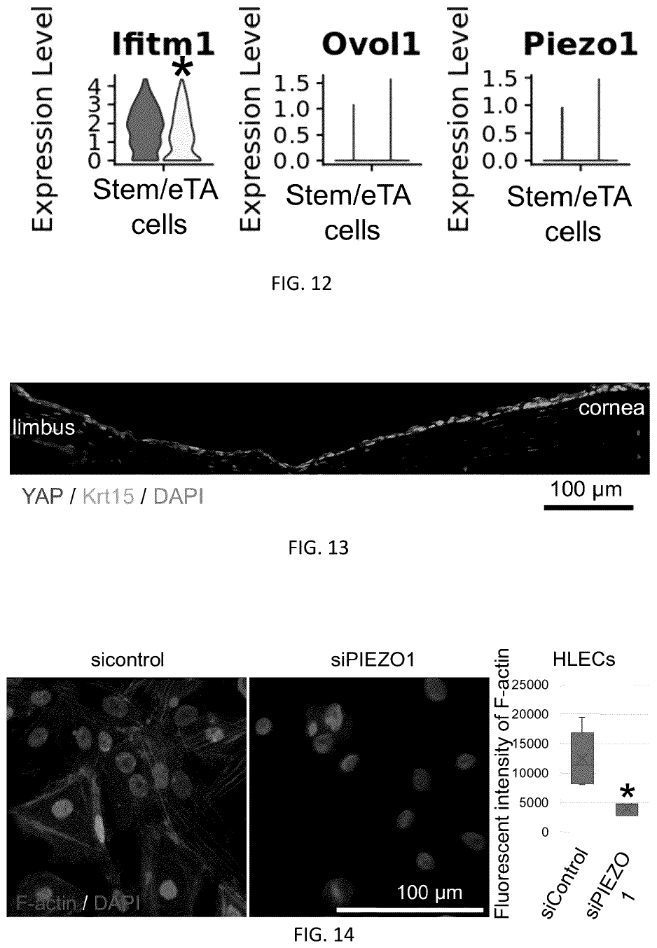

Many corneal diseases (e.g., dry eye disease and diabetic keratopathy) as well as aged corneas are associated with dysfunction of LESCs, leading to impaired activation of stem cells in response to corneal injury^{26, 19-25, 67-70}. scRNA-seq analysis of dry eye disease, diabetic corneas as well as aged mouse corneas found that Ifitm1 expression was reduced in the limbal stem/eTA cell population of these corneas compared to healthy controls, which was accompanied by an increase in Ovol1 expression⁶⁶ (FIG. 12). These observations suggest that downregulation of Ifitm1 may contribute to defective stem cell activation in these diseases.

### Decreasing IFITM1 Enhances Stem/eTA Cell-Enriched Limbal Epithelial Cell Differentiation

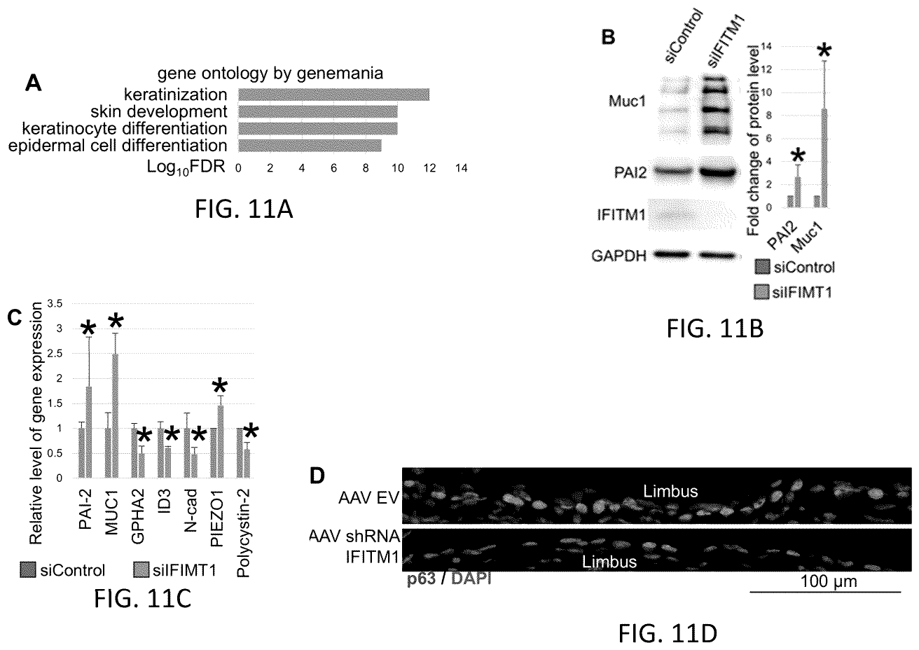

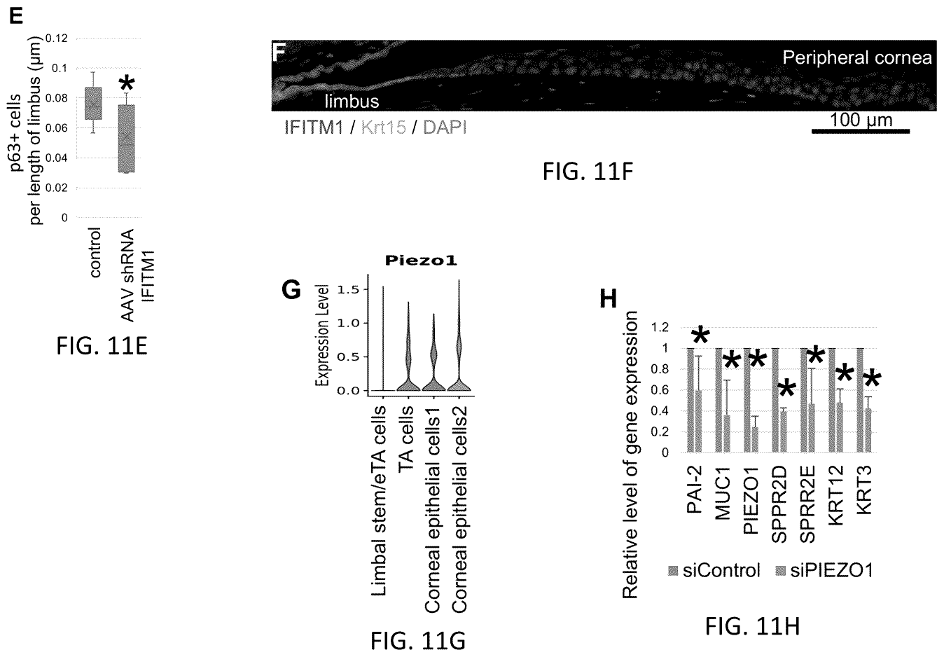

Bulk RNA sequencing of HLECs transfected with siIFITM1 vs siControl identified 945 differentially expressed genes (DEGs). Gene ontology (GO) analysis of the top 50 DEGs indicated an enrichment in GO terms associated with differentiation (FIG. 11A). Knockdown of IFITM1 in HLECs cultures induced the expression of epithelial cell differentiation markers while attenuating the expression of putative limbal epithelial stem cell markers (FIGS. 111B-11C). In vivo, AAV-shRNA Ifitm1 topical application reduced p63+ cells in mouse limbal epithelium (FIGS. 11D-11E). P63 is a putative limbal epithelial stem cell marker^{13,6}. These results suggest that IFITM1 is a negative regulator of stem/eTA cell enriched limbal epithelial cell differentiation. Since IFITM1 expression is markedly higher in limbal epithelium compared to corneal epithelium (FIGS. 2B, 11F).

### PIEZO1 Positively Regulates Differentiation

Many cell membrane proteins (e.g., an ion channel PIEZO1) can sense mechanical stimuli (mechanosensing) and convert these mechanical stimuli, including matrix stiffness (matrix resistance to deformation), into biochemical signals (mechanotransduction). These proteins called mechanosensors can broadly influence cell behavior (e.g., differentiation) via activating downstream transcription factors (e.g., YAP). Recent studies demonstrate that the biomechanics of the cornea and limbus is involved in the functionality as well as maintenance of tissue homeostasis and thus has an impact on vision. It has been shown that the limbus comprises a distinct “softer” (less resistance to deformation) biomechanical microenvironment compared to the relatively “stiffer” (more resistance to deformation) central cornea⁷¹⁻⁷³. This softer mechanical property contributes to maintaining a relatively undifferentiated phenotype, which is a hallmark of LESCs^{71,74}. Conversely, stimulation with stiffer substrates (e.g., when eTA cells centripetally migrate into the stiffer central cornea) can induce cell differentiation^{71, 74-77}. To support this idea, the chemical injury-induced stiffening of the limbal matrix leads to the loss of the undifferentiated state of LESCs⁷⁷. In diabetic corneas, disruption of the basement membrane²⁶ as well as enhanced crosslinking and stiffness in stroma^{78,79} are associated with dysfunction of LESCs. Thus, abnormal mechanical properties of the cornea and limbus can contribute to the development and progression of various corneal diseases. It remains unclear and contradictory how the differentiation is controlled by the signaling pathways involved in mechanotransduction. YAP is a transcription factor, and its nuclear localization is triggered by changes in the cellular environment, including substrate stiffness. Such nuclear localization turns on gene transcription and downstream effects¹⁶. In vitro studies indicate that stiffer substrates induce YAP nuclear translocation and thus corneal epithelial differentiation^{74,80}. We have shown that YAP highly localizes to the nuclei of epithelial cells in the mouse cornea (stiffer) compared to limbus (softer; FIG. 13). Whereas an in vivo study suggests that YAP nuclear localization is irrelevant to limbal/corneal epithelial differentiation and inhibited by more rigid substrates in the central cornea⁸¹.

Our scRNA-seq assay showed that Piezo1 was undetectable in the stem/eTA cell cluster while highly expressed in differentiated corneal epithelial cells (FIG. 11G), suggesting that PIEZO1 is a positive regulator of differentiation. To confirm this idea, knockdown of PIEZO1 reduced the expression of differentiation markers in HLECs (FIG. 11H). Interestingly, we showed that knockdown of IFITM1 increases PIEZO1 while decreased Polycystin-2 (FIG. 11C), an endogenous inhibitor of PIEZO1 activity in variety of non-ocular cell types⁸². These observations indicate that IFITM1 may negatively modulate both the expression and activity of PIEZOL. Overall, these observations support the hypothesis that mechanical stimuli have a role in differentiation. In consistent with this idea, YAP, a transcriptional factor regulated by mechanical stimuli, is highly located in nuclei in corneal epithelial cells, which promotes gene transcription. Whereas in limbal epithelium, the nuclear localization is inhibited (FIG. 13).

PIEZO1 is a positive regulator of actin filament polymerization⁸³. Knockdown of PIEZO1 results in reduced F-actin fibers in limbal epithelial cells (FIG. 14) and such reduction of F-actin fibers is associated with decreased stiffness of epithelial cells¹⁴. Thus, modulation of IFITM1 and PIEZO1 may affect HLEC stiffness and F-actin levels.

### Discussion

Following a corneal injury, the proliferation in limbal epithelium is induced to generate epithelial cells, resulting in re-epithelialization. The induced proliferation is attributed to three strategies: (i) activation of the slow-cycling stem cells to divide, thus producing more early TA cells and expanding stem/early TA cell population; (ii) increasing the number of times these cells can replicate before it becomes postmitotic; and (iii) increasing the efficiency of stem/early TA cell replication by shortening the cell cycle time³. Even though these 3 strategies has been proposed for decades, it remains not completely understood how these processes are regulated since lacking discrete stem cell markers to determine stem cell population. Here, we utilize scRNA seq, which is an ideal approach to characterize rare cell population such as stem cells. We show that an upregulation of IFITM1 after corneal injury contributes to activation of LESCs and thus, the expansion of stem/eTA cells, one of the 3 strategies. Such expansion of stem/eTA cells enhances proliferation in the limbal basal layer. The positive role of IFITM1 in activation of the proliferation of LESCs and expansion of stem/eTA cell population is, at least partially, attributed to inhibiting OVOL1, a negative regulator of epithelial cell proliferation³⁷. Interestingly, Ovol1 expression in limbal epithelium is decreased after corneal injury. Taken together, these observations indicate that IFITM1 plays a positive role in regulating stem/eTA cell expansion in vivo. This observation provides a rational to develop an AAV vector delivering IFITM1 cDNA. Topical application of AAV-IFITM1 cDNA should directly promote the proliferation of LESCs in patients, which may have considerable therapeutic potential in treating diseases associated with the loss of LESCs, such as LSCD.

In vitro, the numbers of limbal epithelial stem/eTA cells can be assessed by colony formation assay, which is a well-established approach to determine proliferative capacity of epithelial cells^{28,50-51}. Based on the proliferative capacity, cells can form 3 different clones: holoclones, meroclones and paraclones 5. Stem/eTA cells have high proliferative capacity and thus can form holoclones in vitro while more differentiated epithelial cells with limited proliferative capacity only can form meroclones and paraclones. Thus, evaluation of holoclone formation can be used to determine the numbers of the stem/eTA cells in vitro. Using this approach, it has been shown that several genes and pathways have roles in altering holonclone formation in limbal epithelial cells. For example, knockdown of IFITM3 reduces formation of all 3 clone types in human limbal epithelial cells¹⁵. We have demonstrated that overexpression of microRNA-103 and microRNA-107 in human epidermal keratinocytes and HLECs give rise to significantly more holoclones⁵¹. We have also shown that pharmacological inhibition of autophagy reduces numbers of holoclones in HLECs²⁸. These observations suggest that these genes and pathways can alter the numbers of stem/eTA cells in cultures. But it remains unclear whether such increased numbers of stem/eTA cells in cell cultures is due to the expansion of stem/eTA cells in cultures or the enhancement of survival of stem/eTA cells in the culture medium. For example, knockdown of Basal cell adhesion molecule (BCAM), a marker of stem/eTA cells in limbal epithelium, decreases cell adhesion and colony formation in limbal epithelial cells⁵², suggesting that such reduction of clone formation may be due to failure of cells attaching to culture dishes and subsequently cell death. Thus, additional in vivo experiments will validate the roles of these genes and pathways in regulating expansion of stem/eTA cell populations.

In vivo observations have shown that many genes and pathways can regulate stem/eTA cell-enriched limbal epithelial cell proliferation. For example, knockout of Adrb2, which is the predominant adrenergic receptor expressed in limbal epithelium, enhances limbal epithelial cell proliferation and the expression of putative limbal stem cell markers via targeting SHH signaling⁵³. We have shown that inhibition of autophagy in vivo suppresses the proliferation in limbal epithelium after wounding²⁸. Overexpression of FOXC1 promotes proliferation in limbal epithelium of Pax6+/− mice following central corneal debridement injury⁵⁴. However, in these studies, because of lacking discrete markers for LESCs, it is impossible to determine the numbers of stem cells using immunostaining or flow cytometry with putative limbal stem cell markers. Thus, it remains unclear whether such alterations of limbal epithelial proliferation are due to alteration of numbers of stem/eTA cells or frequency of quiescent stem/eTA cell cycling to generate their progeny. Here, we took advantage of scRNA seq which is an ideal approach to determine rare cell populations such as stem/eTA cells. This is the first demonstration that scRNA seq can provide direct evidence showing that stem/eTA cell number is regulated by a gene, IFITM1. Our approach can be used to determine the roles of the other potential regulatory genes in stem cell expansion not only in cornea but also in other tissues.

Ovol1 and Ovol2 belong to a transcription factor family, encoding zinc finger proteins homologous to Drosophila melanogaster Ovo. OVOL2 expression increased in posterior polymorphous corneal dystrophy, an autosomal dominant inherited disorder of the corneal endothelium³⁶. In corneal epithelium, OVOL2 is a regulator of the human CEC transcriptional program by repressing genes related to epithelial-to-mesenchymal transition³⁵, suggesting a role in regulating corneal epithelial commitment. In contrast to OVOL2, the function of OVOL1 in cornea was unknown. Interestingly, OVOL1 negatively regulates cell proliferation in a variety of tissues. During the epidermal development, absence of Ovol1 markedly enhances keratinocyte proliferation³⁷. During induction of pluripotent stem cells (iPSCs), knockdown of OVOL1 enhances proliferation in cells with high KLF4 levels⁵⁵. OVOL1 is downregulated in cutaneous squamous cell carcinoma³⁸ and mediates the suppression of proliferation in oral squamous cell carcinoma cells by inhibiting ZEB1 expression³⁹. Here we have shown for the first time that knockdown of Ovol1 induces limbal epithelial cell proliferation, which is involved in the maintenance of corneal homeostasis.

One of the challenges in ex vivo epidermal and corneal epithelial cell therapy is expanding stem cells from a small biopsy. Initially, the expansion of limbal epithelial cells was achieved using a 3T3 fibroblast feeder layer⁵⁶. Recently, human amniotic membrane has been utilized as a platform for expanding limbal epithelial cells^{57,58}, as it replicates essential elements of the limbal epithelial stem cell niche. Since ex vivo expansion of limbal-derived stem cells is useful for treating corneal blindness, we propose that ectopic expression of IFITM1, combined with optimized amniotic membrane protocols, could promote the expansion of limbal stem/eTA cells ex vivo. Moreover, since human epidermal keratinocytes display similar proliferative behavior to limbal epithelial cells, ectopic expression of IFITM1 might also effectively boost the number of epidermal keratinocytes needed for regeneration.

Future studies will be performed to determine whether IFITM1 maintains the undifferentiated state of LESCs via inhibiting the mechanosensory PIEZO1. Without wishing to be bound by any particular theory, we propose that, since stiffer substrates induce epithelial cell differentiation, we propose that downregulation of IFITM1 upregulates PIEZO1, which enhances Ca²⁺ influx and thus promotes differentiation. To test this idea, we will utilize submerged, and 3D raft organotypic cultures of stem/eTA cell-enriched primary HLECs as well as conditional knockout mice. We will examine whether depletion of PIEZO1 will reverse the positive effect of IFITM1 downregulation on limbal epithelial cell differentiation. We will also determine whether overexpression of IFITM1 inhibits differentiation of limbal epithelial cells. To test whether the role of the IFITM1/PIEZO1 axis in differentiation involves the activity of PIEZO1 as an ion channel, we will modulate the endogenous inhibitor of PIEZO1 as well as concentrations of intracellular Ca²⁺, which positively influence differentiation. Finally, we will determine whether blocking mechanotransduction in corneal epithelial cells induces the expression of stem cell markers.

We will also confirm whether enhancing IFITM1 expression induces LESC proliferation and eTA expansion. We will test whether overexpression of IFITM1 and downregulation of OVOL1 promotes the activation of LESCs and thus the expansion of the eTA cell population in healthy young adult mice as well as in aged wild type and diabetic mice, which manifest a reduction of IFITM1 and a defect in LESC activation induced by corneal wounding. We have established AAV-IFITM1 cDNA, an AAV vector that can overexpress IFITM1 in limbal epithelium by topical treatment. We will conduct a rescue experiment to test whether the effect of IFITM1 overexpression on the activation of LESCs is mediated by OVOL1 in vivo. Finally, we will investigate how IFITM1 negatively regulates OVOL1 expression.

### Materials and Methods

### Animal Experiments

Animal procedures were approved by the Northwestern University Animal Care and Use Committee and adherence to the ARVO Statement for the Use of Animals in Ophthalmic and Vision Research. Wt/wt C57BL/6 mice were purchased from The Jackson Laboratory. Mice were housed with free access to food and water in a barrier facility or a confinement facility for AAV transduction. Both male and female mice at 6-8 weeks old were used.

AAV vector with shRNA-IFITM1 was purchased from VectorBuilder and packaged as previously described⁵⁹. To topically apply AAV vector onto mouse corneas, mice were anesthetized and Sul of AAV vector (1×10\hat{ }9gc/ul) were applied topically. The effect was confirmed using another shRNA for IFITM1 (AAV-shRNA Ifitm1 3816; SEQ ID NO: 7).

To generate debridement wounds, as previously described¹⁴, central corneal epithelium was removed using a rotating diamond burr while the peripheral corneal and limbal epithelia remained intact. For alkali burn model, mouse corneas were exposed to 1M NaOH solution for 30 seconds. Three days post injury, mouse corneas were isolated as previously described-L. For the BrdU pulse labeling assay, mice were injected with BrdU (50 g/kg) intraperitoneally 1 hour prior to euthanasia⁶⁰.

### Single Cell RNA Sequencing Analysis

Single-cell isolation from corneal and limbal tissues and scRNA-seq analysis were conducted as previously described^{1-4,18,61}. Data normalization by R Package Seurat was used to account for the differences in the cell number between samples during integration and clustering as previously described^{14, 18, 61, 62}. For data alignment, we selected 4,000 highly variable genes in each data matrix and performed ‘FindIntegrationAnchors’ and ‘IntegrateData’ functions. Next, we performed the clustering using ‘FindClusters’ in Seurat to identify cell type clusters. UMAP of each data set were visualized. The Seurat FindConservedMarkers( ) function was used to obtain a list of marker genes conserved across conditions. These marker genes for each cluster were used to annotate these clusters as specific cell types. Dot Plot was used to visualize conserved cell type markers and show both the expression level and the percentage of cells in a cluster expressing those marker genes. ‘FindMarkers’ was used to identify the differentially expressed genes (DEG) between each sample in each cluster. Functional Annotation Clustering of DEGs was performed in DAVID Functional Annotation Bioinformatics Resources. The scRNA seq data that support the findings of this study are submitted to NCBI GEO database (GSE275678).

### Cell Culture

Epithelial cells were isolated from human corneal buttons obtained from the Eversight eye banks (Ann Arbor, MI, USA) as described before L. Primary human limbal epithelial cells (HLECs) were cultured in keratinocyte SFM media (Life Technologies), and first passage cells were used for experiments. Immortalized limbal epithelial cells, hTCEpi, were cultured in keratinocyte serum free media (Life Technologies) as described previously¹⁸. To knock down gene expression, cells were transfected with a 5 nM siRNA pool against IFITM1, OVOL1 (Invitrogen, Carlsbad, CA, USA) or nontarget control (Invitrogen, Carlsbad, CA, USA) as previously described⁶⁰. Cells incubated with BrdU (10 μM) for 1h were used for the BrdU pulse labeling assay⁶⁰. Total RNA was extracted for RT-qPCR as described below.

### Immunostaining

Eyes were subjected to immunostaining with primary antibodies: IFITM1, OVOL1, Krt15 mouse monoclonal antibody at 1:50 dilution, or BrdU mouse monoclonal antibody (G3G4, Developmental Studies Hybridoma Bank) at 1:10 dilution as previously described¹⁴. Slides were visualized with a Nikon Ti-2 (Nikon, Tokyo, Japan) microscope (Northwestern CAM core)¹⁸. ImageJ was used for cell counting and relative fluorescence analysis.

### Reverse Transcription-Quantitative Polymerase Chain Reaction (RT-qPCR)

Total RNAs were isolated from cells and mouse corneas and purified using a miRNeasy kit (Qiagen, Hilden, Germany). RT-qPCR was performed with a BioRad CFX System using the Roche FastStart Essential DNA Green Master (Roche, Branchburg, NJ, USA) according to the manufacturer's instructions. The primer pairs for RT-qPCR were designed using the IDT PrimerQuest Primer Design Tool (IDT, Coralville, IA, USA).

### RNA-Seq

HLECs were transfected with a 5 nM siRNA pool against IFITM1 (Invitrogen, Carlsbad, CA, USA) or nontarget control (Invitrogen, Carlsbad, CA, USA) as previously described⁶⁰ for 3 days. Then, cells were processed for total RNA isolation using miRNeasy kit (Qiagen, Hilden, Germany). Expression profiling was conducted using a Next-Generation Sequencing platform at Northwestern University Center for Genetic Medicine core. Gene differential expression profiles obtained from RNA-seq analysis were subjected to gene ontology analysis using DAVID.

### Quantification and Statistical Analysis

Unpaired t test and paired t test were performed to determine statistical significance. The data are shown as means±standard deviation (SD). Statistical significance was defined as *p<0.05. All experiments were replicated at least three times.

### REFERENCES FOR EXAMPLE 1

1. Pajoohesh-Ganji, A. & Stepp, M. A. In search of markers for the stem cells of the corneal epithelium. Biol Cell 97, 265-276, doi:10.1042/BC20040114 (2005).
2. Parfitt, G. J. et al. Immunofluorescence Tomography of Mouse Ocular Surface Epithelial Stem Cells and Their Niche Microenvironment. Invest Ophthalmol Vis Sci 56, 7338-7344, doi:10.1167/iovs.15-18038 (2015).
3. Lehrer, M. S., Sun, T. T. & Lavker, R. M. Strategies of epithelial repair: modulation of stem cell and transit amplifying cell proliferation. Journal of cell science 111 (Pt 19), 2867-2875 (1998).
4. Stepp, M. A. & Zieske, J. D. The corneal epithelial stem cell niche. Ocul Surf 3, 15-26 (2005).
5. Cotsarelis, G., Cheng, S. Z., Dong, G., Sun, T. T. & Lavker, R. M. Existence of slow-cycling limbal epithelial basal cells that can be preferentially stimulated to proliferate: implications on epithelial stem cells. Cell 57, 201-209 (1989).
6. Peng, H. et al. microRNA-31/factor-inhibiting hypoxia-inducible factor 1 nexus regulates keratinocyte differentiation. Proc Natl Acad Sci USA 109, 14030-14034, doi:10.1073/pnas.1111292109 (2012).
7. Schermer, A., Galvin, S. & Sun, T. T. Differentiation-related expression of a major 64K corneal keratin in vivo and in culture suggests limbal location of corneal epithelial stem cells. J Cell Biol 103, 49-62, doi:10.1083/jcb.103.1.49 (1986).
8. Notara, M. et al. In sickness and in health: Corneal epithelial stem cell biology, pathology and therapy. Exp Eye Res 90, 188-195, doi:10.1016/j.exer.2009.09.023 (2010).
9. Wolosin, J. M., Budak, M. T. & Akinci, M. A. Ocular surface epithelial and stem cell development. Int J Dev Biol 48, 981-991, doi:10.1387/ijdb.041876jw (2004).
10. Sun, T. T., Tseng, S. C. & Lavker, R. M. Location of corneal epithelial stem cells. Nature 463, E10-11; discussion El1, doi:10.1038/nature08805 (2010).
11. Hayashi, R. et al. N-Cadherin is expressed by putative stem/progenitor cells and melanocytes in the human limbal epithelial stem cell niche. Stem Cells 25, 289-296, doi:10.1634/stemcells.2006-0167 (2007).
12. Lavker, R. M. & Sun, T. T. Epidermal stem cells: properties, markers, and location. Proc Natl Acad Sci USA 97, 13473-13475, doi:10.1073/pnas.250380097 (2000).
13. Pellegrini, G. et al. p63 identifies keratinocyte stem cells. Proc Natl Acad Sci USA 98, 3156-3161, doi:10.1073/pnas.061032098 (2001).
14. Jiang, H. et al. Activation of limbal epithelial proliferation is partly controlled by the ACE2-LCN2 pathway. iScience 27, 110534, doi:10.1016/j.isci.2024.110534 (2024).
15. Altshuler, A. et al. Discrete limbal epithelial stem cell populations mediate corneal homeostasis and wound healing. Cell stem cell 28, 1248-1261 e1248, doi:10.1016/j.stem.2021.04.003 (2021).
16. Collin, J. et al. A single cell atlas of human cornea that defines its development, limbal progenitor cells and their interactions with the immune cells. Ocul Surf 21, 279-298, doi:10.1016/j.jtos.2021.03.010 (2021).
17. Potten, C. S. & Loeffler, M. Stem cells: attributes, cycles, spirals, pitfalls and uncertainties. Lessons for and from the crypt. Development 110, 1001-1020 (1990).
18. Kaplan, N. et al. Single-Cell RNA Transcriptome Helps Define the Limbal/Corneal Epithelial Stem/Early Transit Amplifying Cells and How Autophagy Affects This Population. Invest Ophthalmol Vis Sci 60, 3570-3583, doi:10.1167/iovs.19-27656 (2019).
19. Dong, Y., Peng, H. & Lavker, R. M. Emerging Therapeutic Strategies for Limbal Stem Cell Deficiency. J Ophthalmol 2018, 7894647, doi:10.1155/2018/7894647 (2018).
20. Pellegrini, G. et al. Biological parameters determining the clinical outcome of autologous cultures of limbal stem cells. Regenerative medicine 8, 553-567, doi:10.2217/rme.13.43 (2013).
21. Rama, P. et al. Limbal stem-cell therapy and long-term corneal regeneration. The New England journal of medicine 363, 147-155, doi:10.1056/NEJMoa0905955 (2010).
22. Ljubimov, A. V. & Saghizadeh, M. Progress in corneal wound healing. Prog Retin Eye Res 49, 17-45, doi:10.1016/j.preteyeres.2015.07.002 (2015).
23. Xu, K. & Yu, F. S. Impaired epithelial wound healing and EGFR signaling pathways in the corneas of diabetic rats. Invest Ophthalmol Vis Sci 52, 3301-3308, doi:10.1167/iovs.10-5670 (2011).
24. Kabosova, A. et al. Human diabetic corneas preserve wound healing, basement membrane, integrin and MMP-10 differences from normal corneas in organ culture. Exp Eye Res 77, 211-217 (2003).
25. Xu, K. P., Li, Y., Ljubimov, A. V. & Yu, F. S. High glucose suppresses epidermal growth factor receptor/phosphatidylinositol 3-kinase/Akt signaling pathway and attenuates corneal epithelial wound healing. Diabetes 58, 1077-1085, doi:10.2337/db08-0997 (2009).
26. Saghizadeh, M. et al. Alterations of epithelial stem cell marker patterns in human diabetic corneas and effects of c-met gene therapy. Mol Vis 17, 2177-2190 (2011).
27. Lavker, R. M. & Sun, T. T. Epithelial stem cells: the eye provides a vision. Eye (Lond) 17, 937-942, doi:10.1038/sj.eye.6700575 (2003).
28. Park, J. K. et al. MicroRNAs-103/107 coordinately regulate macropinocytosis and autophagy. J Cell Biol 215, 667-685, doi:10.1083/jcb.201604032 (2016).
29. Wu, X. et al. Productive hepatitis C virus infection of stem cell-derived hepatocytes reveals a critical transition to viral permissiveness during differentiation. PLoS Pathog 8, e1002617, doi:10.1371/journal.ppat.1002617 (2012).
30. Tanaka, S. S., Yamaguchi, Y. L., Tsoi, B., Lickert, H. & Tam, P. P. IFITM/Mil/fragilis family proteins IFITM1 and IFITM3 play distinct roles in mouse primordial germ cell homing and repulsion. Dev Cell 9, 745-756, doi:10.1016/j.devcel.2005.10.010 (2005).
31. Yang, Y. G. et al. Interferon-induced transmembrane protein 1-mediated EGFR/SOX2 signaling axis is essential for progression of non-small cell lung cancer. Int J Cancer 144, 2020-2032, doi:10.1002/ijc.31926 (2019).
32. Kelemen, A. et al. IFITM1 expression determines extracellular vesicle uptake in colorectal cancer. Cell Mol Life Sci 78, 7009-7024, doi:10.1007/s00018-021-03949-w (2021).
33. Zheng, W. et al. Down-regulation of IFITM1 and its growth inhibitory role in cervical squamous cell carcinoma Cancer Cell Int 17, 88, doi:10.1186/s12935-017-0456-0 (2017).
34. Saxena, K., Srikrishnan, S., Celia-Terrassa, T. & Jolly, M. K. OVOL1/2: Drivers of Epithelial Differentiation in Development, Disease, and Reprogramming. Cells Tissues Organs 211, 183-192, doi:10.1159/000511383 (2022).
35. Kitazawa, K. et al. OVOL2 Maintains the Transcriptional Program of Human Corneal Epithelium by Suppressing Epithelial-to-Mesenchymal Transition. Cell Rep 15, 1359-1368, doi:10.1016/j.celrep.2016.04.020 (2016).
36. Chung, D. D. et al. Alterations in GRHL2-OVOL2-ZEB1 axis and aberrant activation of Wnt signaling lead to altered gene transcription in posterior polymorphous corneal dystrophy. Exp Eye Res 188, 107696, doi:10.1016/j.exer.2019.107696 (2019).
37. Nair, M. et al. Ovol1 regulates the growth arrest of embryonic epidermal progenitor cells and represses c-myc transcription. J Cell Biol 173, 253-264, doi:10.1083/jcb.200508196 (2006).
38. Ito, T. et al. Potential role of the OVOL1-OVOL2 axis and c-Myc in the progression of cutaneous squamous cell carcinoma. Mod Pathol 30, 919-927, doi:10.1038/modpathol.2016.169 (2017).
39. Xu, C., Yan, T. & Yang, J. OVOL1 inhibits oral squamous cell carcinoma growth and metastasis by suppressing zinc finger E-box binding homeobox 1. Int J Clin Exp Pathol 12, 2801-2808 (2019).
40. Gipson, I. K. Distribution of mucins at the ocular surface. Exp Eye Res 78, 379-388, doi:10.1016/s0014-4835(03)00204-5 (2004).
41. Tseng, S. C. et al. Correlation of specific keratins with different types of epithelial differentiation: monoclonal antibody studies. Cell 30, 361-372, doi:10.1016/0092-8674(82)90234-3 (1982).
42. Loughner, C. L., Tiwari, A., Kenchegowda, D., Swamynathan, S. & Swamynathan, S. K. Spatiotemporally Controlled Ablation of Klf5 Results in Dysregulated Epithelial Homeostasis in Adult Mouse Corneas. Invest Ophthalmol Vis Sci 58, 4683-4693, doi:10.1167/iovs.17-22498 (2017).
43. Li, J. M. et al. Single-Cell Transcriptomics Identifies a Unique Entity and Signature Markers of Transit-Amplifying Cells in Human Corneal Limbus. Invest Ophthalmol Vis Sci 62, 36, doi:10.1167/iovs.62.9.36 (2021).
44. Catala, P. et al. Single cell transcriptomics reveals the heterogeneity of the human cornea to identify novel markers of the limbus and stroma. Scientific reports 11, 21727, doi:10.1038/s41598-021-01015-w (2021).
45. Lin, J. B. et al. Dry eye disease in mice activates adaptive corneal epithelial regeneration distinct from constitutive renewal in homeostasis. Proc Natl Acad Sci USA 120, e2204134120, doi:10.1073/pnas.2204134120 (2023).
46. Stepp, M. A. Corneal integrins and their functions. Exp Eye Res 83, 3-15, doi:10.1016/j.exer.2006.01.010 (2006).
47. Kurpakus, M. A., Maniaci, M. T. & Esco, M. Expression of keratins K12, K4 and K14 during development of ocular surface epithelium. Curr Eye Res 13, 805-814, doi:10.3109/02713689409025135 (1994).
48. Sartaj, R. et al. Characterization of slow cycling corneal limbal epithelial cells identifies putative stem cell markers. Scientific reports 7, 3793, doi:10.1038/s41598-017-04006-y (2017).
49. Takacs, L. et al. Differentially expressed genes associated with human limbal epithelial phenotypes: new molecules that potentially facilitate selection of stem cell-enriched populations. Invest Ophthalmol Vis Sci 52, 1252-1260, doi:10.1167/iovs.10-5242 (2011).
50. Barrandon, Y. & Green, H. Three clonal types of keratinocyte with different capacities for multiplication. Proc Natl Acad Sci USA 84, 2302-2306, doi:10.1073/pnas.84.8.2302 (1987).
51. Peng, H. et al. microRNA-103/107 Family Regulates Multiple Epithelial Stem Cell Characteristics. Stem Cells 33, 1642-1656, doi:10.1002/stem.1962 (2015).
52. Sasamoto, Y. et al. Limbal BCAM expression identifies a proliferative progenitor population capable of holoclone formation and corneal differentiation. Cell Rep 40, 111166, doi:10.1016/j.celrep.2022.111166 (2022).
53. Zhang, Z. et al. Interference of sympathetic overactivation restores limbal stem/progenitor cells function and accelerates corneal epithelial wound healing in diabetic mice. Biomed Pharmacother 161, 114523, doi:10.1016/j.biopha.2023.114523 (2023).
54. Yang, W. et al. FoxCI activates limbal epithelial stem cells following corneal epithelial debridement. Exp Eye Res 234, 109599, doi:10.1016/j.exer.2023.109599 (2023).
55. Kagawa, H. et al. OVOL1 Influences the Determination and Expansion of iPSC Reprogramming Intermediates. Stem Cell Reports 12, 319-332, doi:10.1016/j.stemcr.2018.12.008 (2019).
56. Pellegrini, G. et al. Long-term restoration of damaged corneal surfaces with autologous cultivated corneal epithelium. Lancet 349, 990-993, doi:10.1016/50140-6736(96)11188-0 (1997).
57. Grueterich, M., Espana, E. & Tseng, S. C. Connexin 43 expression and proliferation of human limbal epithelium on intact and denuded amniotic membrane. Invest Ophthalmol Vis Sci 43, 63-71 (2002).
58. Schwab, I. R. Cultured corneal epithelia for ocular surface disease. Trans Am Ophthalmol Soc 97, 891-986 (1999).
59. Xu, J., Contractor, A. & Zhu, Y. Protocol to map multi-transmitter neurons in the mouse brain using intersectional strategy. STAR Protoc 3, 101907, doi:10.1016/j.xpro.2022.101907 (2022).
60. Kaplan, N. et al. Ciliogenesis and autophagy are coordinately regulated by EphA2 in the cornea to maintain proper epithelial architecture. Ocul Surf 21, 193-205, doi:10.1016/j.jtos.2021.06.006 (2021).
61. Peng, H., Kaplan, N., Liu, M., Jiang, H. & Lavker, R. M. Keeping an Eye Out for Autophagy in the Cornea: Sample Preparation for Single-Cell RNA-Sequencing. Methods Mol Biol, doi:10.1007/7651_2023_502 (2023).
62. Satija, R., Farrell, J. A., Gennert, D., Schier, A. F. & Regev, A. Spatial reconstruction of single-cell gene expression data. Nat Biotechnol 33, 495-502, doi:10.1038/nbt.3192 (2015).
63. Tirosh et al. Science. 2016; 352(6282):189-96. Epub 2016/04/29. doi: 10.1126/science.aad0501.
64. Le et al. Ocul Surf 2018; 16(1):58-69. Epub 2017/11/09. doi: 10.1016/j.jtos.2017.11.002.
65. Warde-Farley et al. Nucleic Acids Res. 2010; 38(Web Server issue):W214-20. Epub 2010/07/02. doi: 10.1093/nar/gkg537.
66. Jiang et al. FASEB J. 2025; 39(10):e70648. Epub 2025/05/15. doi: 10.1096/fj.202500783R.
67. Douvaras et al. Stem Cell Res. 2012; 8(1):109-19. Epub 2011/11/22. doi: 10.1016/j.scr.2011.08.007.
68. Sagga et al. Stem Cell Res. 2018; 33:185-98. Epub 2018/11/16. doi: 10.1016/j.scr.2018.11.001.
69. Moshirfar et al. J Clin Med. 2023; 12(13). Epub 2023/07/14. doi: 10.3390/jcm12134418.
70. Lin et al. The American journal of pathology. 2023; 193(11):1863-78. Epub 2023/08/28. doi: 10.1016/j.ajpath.2023.08.003.
71. Moers et al. Experimental cell research. 2013; 319(12):1889-901. Epub 2013/05/15. doi: 10.1016/j.yexcr.2013.05.002.
72. Lepert et al. Faraday Discuss. 2016; 187:415-28. Epub 2016/04/07. doi: 10.1039/c5fd00152h.
73. Last et al. Micron. 2012; 43(12):1293-8. Epub 2012/03/17. doi: 10.1016/j.micron.2012.02.014.
74. Foster et al. Exp Eye Res. 2014; 127:37-41. Epub 2014/07/06. doi: 10.1016/j.exer.2014.06.020.
75. Jones et al. Stem Cell Res. 2012; 8(3):403-9. Epub 2012/03/06. doi: 10.1016/j. scr.2012.01.001.
76. Eberwein et al. Stem Cells. 2015; 33(3):916-24. Epub 2014/11/21. doi: 10.1002/stem.1886.
77. Gouveia et al. Nature communications. 2019; 10(1):1496. Epub 2019/04/05. doi: 10.1038/s41467-019-09331-6.
78. Ohn et al. Medicine (Baltimore). 2022; 101(36):e30248. Epub 2022/09/11. doi: 10.1097/MD.0000000000030248.
79. Blackburn et al. Front Bioeng Biotechnol. 2019; 7:66. Epub 2019/04/26. doi: 10.3389/fbioe.2019.00066.
80. Gouveia et al. Cells. 2019; 8(4). Epub 2019/04/25. doi: 10.3390/ce11s8040347.
81. Bhattacharya et al. Cell Death Differ. 2023; 30(6):1601-14. Epub 2023/04/25. doi: 10.1038/s41418-023-01156-7.
82. Richardson et al. FEBS J. 2022; 289(15):4447-69. Epub 2021/06/02. doi: 10.1111/febs.16041.
83. Jetta et al. Front Cell Dev Biol. 2023; 11:1198109. Epub 2023/06/09. doi: 10.3389/fcell.2023.1198109.
84. Yu et al. Acta Biochim Biophys Sin (Shanghai). 2021; 53(1):10-8. Epub 2020/11/20. doi: 10.1093/abbs/gmaa112.

### Exemplary Embodiments

Embodiment 1. A method for treating a corneal wound in a subject in need thereof, the method comprising administering a therapeutic agent to the subject that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

Embodiment 2. The method of embodiment 1, wherein the therapeutic agent is topically administered to the eye of the subject.

Embodiment 3. The method of embodiment 1 or 2, wherein the therapeutic agent is administered to the limbal epithelium, conjunctiva, or cornea of the subject.

Embodiment 4. The method of any one of embodiments 1-3, wherein the therapeutic agent comprises a vector that: i) expresses IFITM1 or a variant thereof; and/or ii) expresses a polynucleotide that reduces or eliminates expression of OVOL1.

Embodiment 5. The method of embodiment 4, wherein the vector is a viral vector.

Embodiment 6. The method of embodiment 5, wherein the viral vector is an adenovirus-associated viral vector.

Embodiment 7. The method of any one of embodiments 1-3, wherein the therapeutic agent is a pharmaceutical composition comprising IFITM1 or a variant thereof and one or more of a carrier, excipient, or diluent.

Embodiment 8. The method of any one of embodiments 1-7, wherein the corneal wound is caused at least partly by a physical wound, chemical burn, or disease.

Embodiment 9. The method of embodiment 8, wherein the disease comprises diabetes and the subject exhibits diabetic keratopathy.

Embodiment 10. A method for increasing a population of limbal epithelial stem cells (LESCs) and/or early transit amplifying cells (eTAs) in the limbal epithelium of a subject in need thereof, the method comprising administering a therapeutic agent to the subject that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

Embodiment 11. The method of embodiment 10, wherein the therapeutic agent is topically administered to the eye of the subject.

Embodiment 12. The method of embodiment 10 or 11, wherein the therapeutic agent is administered to the limbal epithelium, conjunctiva, or cornea of the subject.

Embodiment 13. The method of any one of embodiments 10-12, wherein the therapeutic agent comprises a vector that: i) expresses IFITM1 or a variant thereof, and/or ii) expresses a polynucleotide that reduces or eliminates expression of OVOL1.

Embodiment 14. The method of embodiment 13, wherein the vector is a viral vector, such as an adenovirus-associated viral vector.

Embodiment 15. The method of any one of embodiments 10-12, wherein the therapeutic agent is a pharmaceutical composition comprising IFITM1 or a variant thereof and one or more of a carrier, excipient, or diluent.

Embodiment 16. The method of any one of embodiments 10-15, wherein the corneal wound is caused at least partly by a physical wound, chemical burn, or disease.

Embodiment 17. The method of embodiment 16, wherein the disease comprises diabetes and the subject exhibits diabetic keratopathy.

Embodiment 18. The method of any one of embodiments 4-9 or 13-17, wherein the IFITM1 or variant thereof has an amino acid sequence having at least 85%, 90%, 95%, 96%, 97%, 98%, 99%, or 100% identity to SEQ ID NO: 1.

Embodiment 19. The method of any one of embodiments 4-6, 8-9, 13-14, or 16-18, wherein the vector comprises a heterologous nucleic acid sequence having at least 85%, 90%, 95%, 96%, 97%, 98%, 99%, or 100% identity to SEQ ID NO: 4.

Embodiment 20. An adeno-associated viral (AAV) vector comprising a heterologous nucleic acid sequence encoding IFITM1 or a variant thereof, operably linked to a regulatory sequence(s) which directs expression of the IFITM1 or the variant thereof.

Embodiment 21. The AAV of embodiment 20, wherein the IFITM1 or variant thereof has an amino acid sequence having least about 85%, 90%, 95%, 96%, 97%, 98%, 99%, or 100% identity to SEQ ID NO: 1.

Embodiment 22. The AAV of embodiment 20 or 21, wherein the AAV comprises a heterologous nucleic acid sequence having at least about 85%, 90%, 95%, 96%, 97%, 98%, 99%, or 100% identity to SEQ ID NO: 4.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating a corneal wound in a subject in need thereof, the method comprising administering a therapeutic agent to the subject that: i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

2. The method of claim 1, wherein the therapeutic agent is topically administered to the eye of the subject.

3. The method of claim 1, wherein the therapeutic agent is administered to the limbal epithelium, conjunctiva, or cornea of the subject.

4. The method of claim 1, wherein the therapeutic agent comprises a vector that: i) expresses IFITM1 or a variant thereof, and/or ii) expresses a polynucleotide that reduces or eliminates expression of OVOL1.

5. The method of claim 4, wherein the vector is a viral vector.

6. The method of claim 5, wherein the viral vector is an adenovirus-associated viral vector.

7. The method of claim 1, wherein the therapeutic agent is a pharmaceutical composition comprising IFITM1 or a variant thereof and one or more of a carrier, excipient, or diluent.

8. The method of claim 1, wherein the corneal wound is caused at least partly by a physical wound, chemical burn, or disease.

9. The method of claim 8, wherein the disease comprises diabetes and the subject exhibits diabetic keratopathy.

10. A method for increasing a population of limbal epithelial stem cells (LESCs) and/or early transit amplifying cells (eTAs) in the limbal epithelium of a subject in need thereof, the method comprising administering a therapeutic agent to the subject that:
(i) results in an increase in concentration of IFITM1 (Interferon Induced transmembrane Protein 1) in the limbal epithelium of the subject compared to the concentration of IFITM1 in the limbal epithelium of the subject prior to administering the therapeutic agent; and/or
(ii) results in a decrease in concentration of OVOL1 (Ovo Like Zinc Finger 1) in the limbal epithelium of the subject compared to the concentration of OVOL1 in the limbal epithelium of the subject prior to administering the therapeutic agent.

11. The method of claim 10, wherein the therapeutic agent is topically administered to the eye of the subject.

12. The method of claim 10, wherein the therapeutic agent is administered to the limbal epithelium, conjunctiva, or cornea of the subject.

13. The method of claim 10, wherein the therapeutic agent comprises a vector that: i) expresses IFITM1 or a variant thereof; and/or ii) expresses a polynucleotide that reduces or eliminates expression of OVOL1.

14. The method of claim 13, wherein the vector is a viral vector, such as an adenovirus-associated viral vector.

15. The method of claim 10, wherein the therapeutic agent is a pharmaceutical composition comprising IFITM1 or a variant thereof and one or more of a carrier, excipient, or diluent.

16. The method of claim 10, wherein the corneal wound is caused at least partly by a physical wound, chemical burn, or disease.

17. The method of claim 16, wherein the disease comprises diabetes and the subject exhibits diabetic keratopathy.

18. An adeno-associated virus (AAV) comprising a heterologous nucleic acid sequence encoding IFITM1 or a variant thereof, operably linked to a regulatory sequence(s) which directs expression of the IFITM1 or the variant thereof.

19. The AAV of claim 18, wherein the IFITM1 or variant thereof has an amino acid sequence that having at least 85%, 90%, 95%, 96%, 97%, 98%, 99%, or 100% identity to SEQ ID NO: 1.

20. The AAV of claim 18, wherein the AAV comprises a heterologous nucleic acid sequence having at least 85%, 90%, 95%, 96%, 97%, 98%, 99%, or 100% identity to SEQ ID NO: 4.


SEQUENCE LISTING (7 sequences)

SEQ ID NO: 1 - protein, 125 aa, Homo sapiens
MHKEEHEVAVLGPPPSTILPRSTVINIHSETSVPDHVVWSLFNTLFLNWCCLGFIAFAYSVKSRDRKMVGDVTGAQAYASTAKCLNIWALILGILMTIGFILLLVFGSVTVYHIMLQIIQEKRGY

SEQ ID NO: 2 - RNA, 668 nt, Homo sapiens
  source 1..668: mol_type = mRNA
gaaacgacaggggaaaggaggtctcactgagcaccgtcccagcatccggacaccacagcggcccttcgctccacgcagaaaaccacacttctcaaaccttcactcaacacttccttccccaaagccagaagatgcacaaggaggaacatgaggtggctgtgctggggccaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtga

SEQ ID NO: 3 - DNA, 2988 bp, Homo sapiens
  source 1..2988: mol_type = genomic DNA
gcactttaagacttcccgagcggcggcggggacgccagtcgagccgggagacgcttacctgccgcttccccgcgccgcccggtgcacctggccgcaagggacctcgttctcagggaagacggcgacattccgcggaggtggaaccgccgcgcgccgtccgggctcggaccttccccggaacgtgggggcgccttagcgactccttccctgttgtgcccccgttcccggcgttcagcccggccccgcaaaggtgggacggctcccggcttcagttacggaagcggcccgtgtccagcgacgagggttcgaaaatgccccgcgcgttcctggtgaagaagccgtgcgtctccacgtgcaagaggaactggagcgagctccccgacgaggagcgcggcgagatctacgtgccagtcagcctgggcttctgcccaccacagccctaccgggagccggaaccctctgtggccgaacccccttcctgcccgctggctttgaacatgagccttcgagactctagctacagcatggcccccgggccctgtgtggtggcccagctgccctctgaagacatgggccacttgacagacccccagagcagagaccatggcttcctgcgcaccaagatgaaggtgacccttggggacagtcccagtggagacctgttcacctgccgtgtctgccagaaggccttcacctaccagcgcatgctgaaccgccacatgaagtgtcacaacgacgtcaagaggcacctctgcacgtactgcgggaagggcttcaatgacaccttcgacctcaagagacacgtccgaactcacactggcgtgcggccctacaagtgcagcctgtgtgacaaggccttcacgcagcgctgctctctggagtctcacctcaagaagatccatggtgtgcagcagaagtacgcgtacaaggagcggcgggccaagctgtacgtgtgtgaggagtgcggctgcacatctgagagccaggagggccacgtcctgcacctgaaggagcaccaccctgacagcccgctgctgcgcaagacctccaagaaggtggccgtggcactacagaacactgtcacttccctgctgcagggcagcccccacctgtgagtggctcgagccctgggggtgctcctggaagccccaagagcatccaggattgcctcccagctgcctggccagcccaccctcctgcaacctctcacccgaacaccagtgatcaggactggagcccccgtgccttggtctcccccctgggcacacgtgctcactcaggcccagcaatgacctctgctcatttttgcatttttgacttatgggccgaggctgttctgagcctgggaagatgtacctatgtcaagagaagggatgaggccaaggctgccttcaattagaagcagccgcccacagagacaggcactgtgtgcctggcagcaggacttcctacccagaggaggttcgagctaggatcccactgcccccgcctctcagcacagggcaggggctgcaggtccccagtggacatcagagtcaaaagcactggcaaagggtacccctgcaaacaactgtggtgggggctggcagcagaccccccacctggcagggcttctaatgctcagggttctggagggctctgtccttccggcaaggagaggcacacatgtgtgcccagccgtgtgtgtgcgtgtgcttgtgtgtgtgcactgctgtgtgtgtgtgcacgcacaggaagcctttccacatatcacctcatttctaagaaataaactacaaggtgccaagaaggttttatttccttttattttttaaagatgacaaatgtacagatgttaatatatttttggtgccaatggcgatgtttttaagagtgggatggagctggcttttctccattcccgtgcgcttctatttatcctggacatttcaaacctcctctgtgccttggctctgggcgggggctgccccaacccacccccgttctttgtacgtgctgagacagccactagaagatcttcctccagcggcgccctggacggctgctcctgcgaacagcccatggcatcttctgctcttccctcccggctctgccctgcacatcctgttgagccaagccccagtgacccggagagctggcctgatgctgagagtgtgtcctcctggggctttagggggcaggaaggtgggacgaatgacgatgcccatccactacctgaagcactaggacactcttgcagggccaggctggaagaccggcccttttcttggttgagtcaaaagccttagcacagtggcaaaaaatgggacagaatgatgaccagcacctcagaaacttccagagggaggagaggatttgatggctaccaaattgtatctgttgccttttttctgactttttcacctgaccaggctggggtttggagtggctgtggggagacccgtcctggctggctggctggctcccttgctcccttgctgcagctgggaaaggggttctgggtgtaaagaggtgtgcgtcttgtgggccaaagggaaaaacaggcaggggtcagagccagcctgccagaggcaaatgcaaaagaggtccccagaacacagccagctgggcagccccttaaagccaaaccccacctgaagcagaaccactttggcctcccctgcccaaaatgggtagtgtctacacgtccccgggctcaggctcaggcccagccctgggctgacctgagaggaaggctccttcctggactgccctctgaaatgtgtatagattgattctaaaatctcttgtttcacttgactttagagtgtctgggacgctgctgtattctgaaagtcacatagcacacagtaatgttatctggaagctctgtttttgtttacatttctgtatccctgggttgactgccaatccgaggccgtcatgaagctctgtgttgtctgttttattttataaccttcctctcaactattaaaattagagatctaa

SEQ ID NO: 4 - DNA, 5556 bp, synthetic construct
  source 1..5556: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgcacaaggaggaacatgaggtggctgtgctggggccaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttacggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 5 - protein, 267 aa, Homo sapiens
MPRAFLVKKPCVSTCKRNWSELPDEERGEIYVPVSLGFCPPQPYREPEPSVAEPPSCPLALNMSLRDSSYSMAPGPCVVAQLPSEDMGHLTDPQSRDHGFLRTKMKVTLGDSPSGDLFTCRVCQKAFTYQRMLNRHMKCHNDVKRHLCTYCGKGFNDTFDLKRHVRTHTGVRPYKCSLCDKAFTQRCSLESHLKKIHGVQQKYAYKERRAKLYVCEECGCTSESQEGHVLHLKEHHPDSPLLRKTSKKVAVALQNTVTSLLQGSPHL

SEQ ID NO: 6 - DNA, 5353 bp, synthetic construct
  source 1..5353: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcacaatcaacatgcctgagatctcgagatctcaggcatgttgattgtgtttttgaattccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcag

SEQ ID NO: 7 - DNA, 5353 bp, synthetic construct
  source 1..5353: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcctgaacatcagctccctgttctcgagaacagggagctgatgttcaggtttttgaattccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcag